(19) United States
(12) Patent Application Publication
(10) Pub. No.: US 2017/0128592 A1
(43) Pub. Date: May 11, 2017

(54) TARGETED POLYMERIC NANO-COMPLEXES AS DRUG DELIVERY SYSTEM

(71) Applicant: Indian Institute of Technology, Bombay, Powai (IN)

(72) Inventors: Radhika POOJARI, Powai (IN); Dulal Panda, Powai (IN); Rohit Srivastava, Powai (IN)

(21) Appl. No.: 15/276,810

(22) Filed: Sep. 27, 2016

(30) Foreign Application Priority Data
Oct. 1, 2015 (IN) 3738/MUM/2015

(51) Int. Cl. A61K 9/16; A61K 9/51; A61K 31/09; A61K 31/565

(52) U.S. Cl. CPC A61K 47/48869; A61K 47/482; A61K 47/48215; A61K 47/48561; A61K 31/09; A61K 31/565; A61K 9/5192

(57) ABSTRACT

The present invention provides targeted polymeric nano-complexes for delivery of drugs such as anti-mitotic agents or anti-cancer agents. The present invention also provides a process for the preparation of such targeted nano-complexes.

### FIELD OF THE INVENTION

The present invention relates to targeted polymeric nano-complexes for delivery of drugs such as anti-mitotic agents or anti-cancer agents. The present invention also relates to a process for the preparation of such targeted nano-complexes.

### BACKGROUND OF THE INVENTION

Hepatocellular carcinoma (HCC) is the third leading cause of liver cancer death worldwide. Until now no systemic treatment of HCC has been effective and its clinical therapy remains a major challenge. The survival of HCC cells has been mainly attributed to the inefficiency of drug penetration into solid tumors, poor selectivity for achieving a therapeutic effect and impaired apoptosis machinery. [Llovet, J. M.; Burroughs, A.; Bruix, J. Hepatocellular carcinoma. Lancet 2003, 362, 1907-1917; Yang, J. D.; Roberts, L. R. Hepatocellular carcinoma: a global view. Nat. Rev. Gastroenterol. Hepatol. 2010, 7, 448-458] Therefore, it is important to identify tumor-specific targets and to develop new therapeutics, which can selectively treat HCC.

Microtubules are established as drug targets for various types of tumors. Microtubule targeting agents interfere with mitotic spindle formation in cancer cells, arrest cells in mitosis and induce apoptosis. Certain microtubule destabilizing agents such as combretastatin A4 (CA4) and 2-methoxyestradiol (2 ME) have been shown to have potent anti-proliferative, anti-invasive, anti-angiogenic and anti-metastatic activities indicating that these compounds utilize diverse mechanisms to be effective for solid tumors. Presently, these compounds are under clinical trials in the form of pro-drugs, its analogues, combination chemotherapeutics and targeting theranostics for various types of advanced cancers. Key limitations of these drugs (CA4 and 2 ME) are the tedious isolation procedures, poor aqueous solubility, short half-life, poor bioavailability and adverse effects of cremophor EL/ethanol solvents used in formulations. In addition, non-specific delivery causing non-tumor cells killing, systemic toxicity and neurotoxicity hinder the therapeutic outcome in clinical settings.

The development of drug resistance further reduces the efficacy of these drugs. Hence, nano-sized technology for delivery of such drugs could be a promising approach. To improve the aqueous solubility, safety and pharmacokinetic properties of drugs such as CA4 or 2 ME, several delivery systems such as polymeric micelles, liposomes, nanosuspension, nanocrystals, dendrimers, magnetic nanoparticles, fullerene (C60) and peptidic macromolecules have been attempted. However, these delivery systems have various disadvantages such as insufficient drug loading capacity, high production cost, drug expulsion, fusion of encapsulated drug or molecules, short half-life and less stability.

Targeted polymeric nanocarriers based biocompatible, targeted, controlled drug delivery systems may be considered to be innovative treatment strategies for liver malignancies. To reach throughout the solid tumour cells, nanocarriers should extravasate from blood circulation to tumour interstitial space and diffuse evenly. Efficient accumulation in tumour cells and ligand-receptor mediated active tumour targeting are the important avenues for increasing the treatment efficiency and to reduce the toxicity of therapeutic agents. Epideinial growth factor receptor (EGFR) overexpressing tumours are associated with an aggressive stage and poor prognosis in terms of survival including solid tumours like HCC. It also involves tumour proliferation, progression and drug resistance. EGFR (HERO) is a member of the ErbB family of receptors. EGFR signaling in tumour cells influences the neoplastic growth.

The present invention, therefore, deals with a novel approach of providing polymeric nanocomplexes conjugated with anti-EGFR targeting moiety as a potent drug delivery system. The nanocomplexes of the present invention effectively perturbs the organization and stability of microtubules in cancer cells such as Huh7 cells, increases phospho-Histone H3 expression and potently inhibits the proliferation as well as the migration of Huh7 cells. The combinatorial therapy of the nanocomplexes markedly enhanced the anticancer activity.

### SUMMARY OF THE INVENTION

In one aspect, the present invention provides targeted nano-complexes comprising:
a hydrophobic polymer core encapsulating at least one drug inside the core;
a hydrophilic polymer conjugated to said core; and
an anti-epidermal growth factor receptor (anti-EGFR) targeting moiety conjugated to the hydrophilic polymer.

In another aspect, the present invention provides a process for the preparation of targeted nano-complexes comprising the steps of:
a) conjugating a hydrophobic polymer with a hydrophilic polymer to form a diblock co-polymer;
b) precipitating said diblock co-polymer in ice-cold diethyl ether/methanol solution and drying said diblock co-polymer under vacuum;
c) dissolving said diblock co-polymer and drug in a solvent and emulsifying it in a surfactant solution using an ultrasonic processor to form an oil-in-water emulsion;
d) stirring the resulting emulsion and subjecting it to solvent evaporation using rotary evaporator to form an aqueous suspension of drug encapsulated nanoparticles;
e) centrifuging said nanoparticles at 20,000×g for 20 minutes at 4° C.;
f) washing said nanoparticles with water and re-suspending it in phosphate buffered saline; and
g) conjugating drug encapsulated nanoparticles with an anti-epidermal growth factor receptor (anti-EGFR) targeting moiety to form the nano-complex.

### OBJECTS OF THE INVENTION

It is an object of the present invention to provide targeted polymeric nano-complexes for delivery of drugs such as anti-mitotic agents or anti-cancer agents. The polymeric nano-complexes are conjugated with an anti-EGFR targeting moiety such an anti-EGFR antibody to target over-expressed epidermal growth factor receptor (EGFR) cancer cells.

Another object of the present invention is to provide polymeric nanocarriers based biocompatible, stable and controlled drug delivery systems.

Another object of the present invention is to provide targeted drug delivery system for combinatorial delivery of drugs.

Another object of the present invention is to provide accelerated cellular internalization of the targeted nano-complex. M cancer cells by anti-EGFR antibody active targeting.

Another object of the present invention is to provide targeted nano-complexes which inhibit phosphor-EGFR expression, increase phospho-Histone H3 expression, depolymerize microtubules, produce spindle abnormalities, stall mitosis and induce apoptosis in cancer cells.

Another object of the present invention is to provide targeted nano-complexes which inhibit the proliferation as well as the migration of cancer cells.

Another object of the present invention is to provide increased stability and half-plasma life of the targeted nano-complexes in order to decrease the phagocytic uptake and improve bioavailability of the drug encapsulated in the nano-complexes.

Yet another object of the present invention is to provide enhanced anticancer efficacy of targeted nano-complexes in comparison to the non-targeted counterparts against EGFR overexpressed cancer cells, with remarkable therapeutic potency exhibited by combinatorial targeted drug delivery system.

### BRIEF DESCRIPTION OF THE DRAWINGS

The following detailed description of preferred embodiments of the invention will be better understood when read in conjunction with the appended drawings. For the purpose of illustrating the invention, there are shown in the drawings, embodiments which are presently preferred. It should be understood, however, that the invention is not limited to the precise arrangements and instrumentalities shown in the drawings.

FIG. 1: (FIG. 1A) A schematic representation of the synthesis and preparation of combretastatin A4 (CA4) or 2-methoxyestradiol (2-ME) encapsulated in the core of PLGA-b-PEG co-polymeric nano-complex via an emulsion-solvent evaporation method, and targeted with a cetuximab-functionalized corona, (FIG. 1B) Internalization of cetuximab-nanocomplexes to liver cancer cell with EGFR overexpression.

FIG. 2: (FIG. 2A) NMR (400 MHz) spectra of synthesized PLGA-b-PEG diblock co-polymer in the presence of heterobifunctional linker H₂N-PEG₂₀₀₀-COOH in CDCl₃. Inset showing PEG conjugation at 3.65 ppm. (FIG. 2B) Representative FEG-TEM and FEG-SEM images of Cet-PLGA-b-PEG NP, Cet-PLGA-b-PEG-CA4 NP and Cet-PLGA-b-PEG-2 ME NP. (FIG. 2C) XPS showing survey scan of the surfaces of PLGA-b-PEG NP vs Cet-PLGA-b-PEG NP with elemental ratio (%), and enlarged view and high resolution elemental scans N 1s, S 2p, C 1s of Cet-PLGA-b-PEG NP (below). (FIG. 2D) DLS showing the particle size distribution of cetuximab-nanocompexes.

FIG. 3: (FIG. 3A) MALDI-TOF MS showing the mass spectra of PLGA-b-PEG NP, Cetuximab, Cet-PLGA-b-PEG-CA4 NP and Cet-PLGA-b-PEG-2 ME NP. (FIG. 3B) SDS-PAGE showing conjugation confirmation of Cet-NP in Coomassie Brilliant Blue stained bands. (FIG. 3C) MALDI-TOF MS/MS fingerprint spectrum of the Trypsin digested Cet-PLGA-b-PEG NP, Mascot score histogram (Inset) and peptide identification (Row below).

FIG. 4: CLSM images and flow cytometric analysis showing the cellular uptake targeting efficiencies of PLGA-b-PEG NP-Rhodamine B (Rh B) and Cet-PLGA-b-PEG NP-Rh B (100 μg/mL) at 3 hr and 24 hr in the EGFR negative Hep G2 (FIG. 4A and FIG. 4B) and the EGFR overexpressing Huh7 (FIG. 4C and FIG. 4D) cells. Scale bars are 50 μm.

FIG. 5: CLSM images of Huh7 cells showing the intracellular distribution of PLGA-b-PEG NP-Rh B and Cet-PLGA-b-PEG NP-Rh B (100 μg/mL) at 24 hr. Nuclei were stained blue with Hoechst. (FIG. 5A) EGFR cell membrane localization (anti-EGER) and (FIG. 5B) microtubule organization (anti-α-tubulin) are seen as green fluorescence, and the nanocomplexes (NP) as red fluorescence in the cells. Scale bars are 10 μm. (FIG. 5C) Representative ultrastructural TEM images of Huh7 cells in untreated (control), PLGA-b-PEG NP, Cet-PLGA-b-PEG NP, Cet-CA4 or −2 ME nano-complexes and Cet-combinatorial nano-complex treated cells showing cellular internalization at 24 hr. Oval dotted circle shows the disruption of the cell membrane integrity, penetration of the nano-complexes inside the cell and round dotted circle in the inset shows internalized nanoparticles at a higher magnification. Nucleus=N. Scale bars are 2 μm.

FIG. 6: Immunofluorescence analysis of the effects of the nanocomplexes and free drugs on pEGFR and microtubules in Huh7 cells. Huh7 cells were treated with (FIG. 6A) 15 nM CA4 or (FIG. 6B) 5 μM 2 ME, and their respective nanocomplexes as well as Cet-combinatorial nano-complexes (15 nM+5 μM) for 48 hr. Cells were fixed and stained for anti-pEGFR and anti-α-tubulin seen as green and red (microtubules) fluorescence, respectively. Chromosomes/nuclei were stained blue with Hoechst. Scale bars are 10 μm.

FIG. 7: Representative Western blot analysis showing (FIG. 7A) inhibition of EGFR phosphorylation in Huh7 cells at 48 hr on treatment with various nanocomplexes at indicated concentrations. P/T represents phosphorylated/total EGFR ratio. Membranes were reprobed for β-actin as the loading control. (FIG. 7B) Polymerized tubulin was differentially sedimented from soltible tubulin in cell lysates prepared in microtubule stabilizing buffer and were analyzed for the microtubule-depolymerizing effects of the indicated treatment groups using anti-α-tubulin antibody.

FIG. 8: Differential cytotoxicities of PLGA-b-PEG NP, Cet-PLGA-b-PEG NP in Hep G2 and Huh7 cells (FIG. 8A), CA4 (FIG. 8B), 2 ME (FIG. 8C) and their respective nanocomplexes at different concentrations in Huh7 cells after 48 hr treatment. Percentage of growth inhibition of each data point is represented as mean±SD of three independent experiments.

FIG. 9: Apoptosis induction of various nano-complexes treated Huh7 cells at IC₅₀ concentrations for 48 hr by FACS analysis. Q2, the upper right quadrant, indicates the percentage of the late apoptotic cells (PI stained cells); Q4, the lower right quadrant, indicates the percentage of early apoptotic cells (Annexin V stained positive cells); Q1, the upper left quadrant, indicates dead cells and Q3, the lower left quadrant, indicates live cells. Data are mean±SD of three independent experiments.

FIG. 10: Effects of nano-complexes and the free drugs on Huh7 cell migration analysis. Representative images of wound closure at different (0, 4 and 8 hr) time points upon treatment with 15 nM CA4 or 5 μM 2 ME and their respective nanocomplexes as well as Cet-combinatorial nano-complexes (15 nM+5 μM). Scale bars are 50 μm.

FIG. 11: (FIG. 11A) Representative enlarged ultrastructural TEM image of Huh7 cells in Cel-combinatorial nano-complex treated cells showing cellular internalization at 24 hr. Round dotted circle shows internalized nanoparticles at a higher magnification. Scale bar is 2 μm. (FIG. 11B) Enlarged version of FIG. 11A. Round dotted circle shows internalized nanoparticles at a higher magnification. Scale bar is 2 μm. (FIG. 11C) Enlarged version of FIG. 11B. Round dotted circle shows internalized nanoparticles at a higher magnification. Scale bar is 1 μm.

FIG. 12: Representative western blot analysis showing EGFR expression protein levels in L929, NIH3T3 normal cells and Hep G2, Huh7 HCC cells at 48 hr. Membranes were reprobed for β-actin as the loading control.

FIG. 13: CLSM images showing the effect of 15 nM CA4 or 5 μM 2 ME and their respective nanocomplexes as well as Cet-combinatorial nano-complexes (15 nM+5 μM) on the pHistone H3 nuclear expression in Huh7 cells at 48 hr. Nuclei were stained blue with Hoechst and anti-pHistone H3 (Ser 10), the nuclear marker is seen as green fluorescence. Scale bars are 10 μm.

FIG. 14: Biocompatibility of PLGA-b-PEG NP and Cet-PLGA-b-PEG NP in normal cells L929 (FIG. 14A) and NIH3T3 (FIG. 14B) at different concentrations after 48 hr treatment. Percentage of growth inhibition of each data point is represented as mean±SD of three independent experiments.

### DESCRIPTION OF THE INVENTION

In describing and claiming the invention, the following terminology will be used in accordance with the definitions set forth below. Unless defined otherwise, all technical and scientific terms used herein have the same meaning as commonly understood by one of ordinary skill in the art to which this invention pertains. In the case of conflict, the present document, including definitions will control.

As used herein, the singular forms “a,” “an,” and “the” include plural reference unless the context clearly dictates otherwise.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “comprising” “including,” “having,” “containing,” “involving,” and the like are to be understood to be open-ended, i.e., to mean including but not limited to.

Thus, before describing the present invention in detail, it is to be understood that this invention is not limited to particularly exemplified systems or process parameters that may of course, vary. It is also to be understood that the terminology used herein is for the purpose of describing particular embodiments of the invention only, and is not intended to limit the scope of the invention in any manner.

The use of examples anywhere in this specification including examples of any terms discussed herein is illustrative only, and in no way limits the scope and meaning of the invention or of any exemplified term. Likewise, the invention is not limited to various embodiments given in this specification.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “comprising” “including,” “having,” “containing,” “involving,” and the like are to be understood to be open-ended, i.e., to mean including but not limited to.

In accordance with the present invention, there is provided targeted nano-complexes comprising:
a hydrophobic polymer core encapsulating at least one drug inside the core;
a hydrophilic polymer conjugated to said core; and
an anti-epidermal growth factor receptor (anti-EGFR) targeting moiety conjugated to the hydrophilic polymer.

The hydrophobic polymer used in the present invention is poly (lactic-co-glycolic acid) [PLEA], 50/50 PLGA, 65/35 PLGA, 75/25 PLGA, 85/15 PLGA or its derivatives. The hydrophobic polymer used in the present invention may be used for site-specific targeting and controlled-release of encapsulated drug. It may encapsulate either hydrophobic drug or hydrophilic drug. It has non-antigenic properties and is easy-to-scale up in the manufacturing process.

The hydrophilic polymer used in the present invention is polyethylene glycol (PEG). The hydrophilic polymer used in the present invention provides stealth surface effects, increases the stability and half-plasma life of the nanocomplexes in order to decrease the phagocytic uptake and improves the bioavailability of the drug. It also decreases the non-specific bio-fouling of the nano-complexes. The hydrophilic polymer forms a corona on the hydrophobic polymer core.

Both the hydrophobic and hydrophilic polymer used in the nano-complexes of the present invention is biodegradable and biocompatible.

The anti-epidermal growth factor receptor (anti-EGFR) targeting moiety used in the present invention is an anti-EGFR antibody, an anti-EGFR antibody-like molecule, an anti-EGFR Fe portion, an anti-EGFR Fab, an anti-EGFR Fab₂, an anti-EGFR ScFv, an anti-EGFR single domain antibody or an anti-EGFR nanobody or an anti-EGFR ligand antibody or an anti-EGFR ligand antibody fragment or its variants or fragments thereof Preferably, the anti-EGFR targeting moiety is cetuximab, pinituniumab, zalutumumab, nimotuzumab, matuzumab or its variants and/or its fragments. More preferably, the anti-EGFR targeting moiety is an anti-EGFR antibody such as cetuximab or its variants and/or its fragments.

Monoclonal antibodies such as cetuximab can be used as EGFR antagonists for tumor specific chemotherapeutics delivery. Cetuximab is a chimeric monoclonal antibody having two N-linked carbohydrate sites on both heavy chains and clinically approved for a broad panel of cancers. It has a long half-life of 70-100 hr. Cetuximab binds with high specificity to the extracellular domain of the human EGFR. Binding of cetuximab with EGFR induces internalization of EGFR leading to down-regulation of EGFR. This also leads to the activation of cytotoxic immune effector cells towards EGFR-expressing tumor cells.

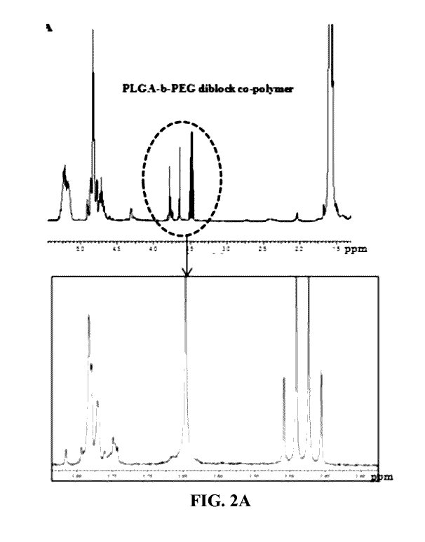

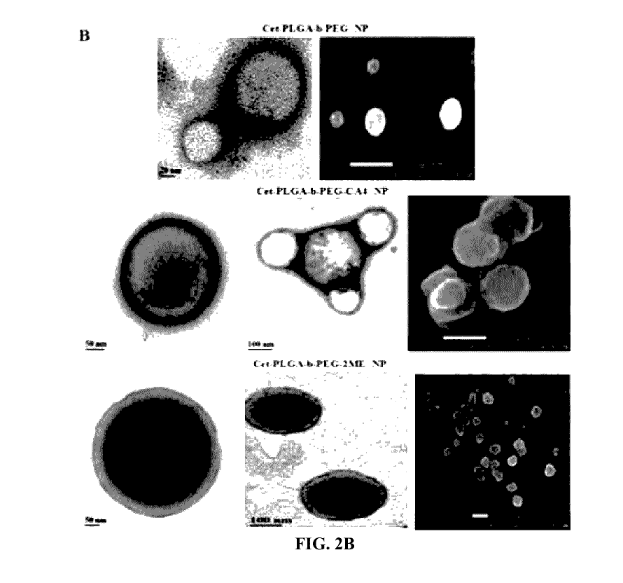

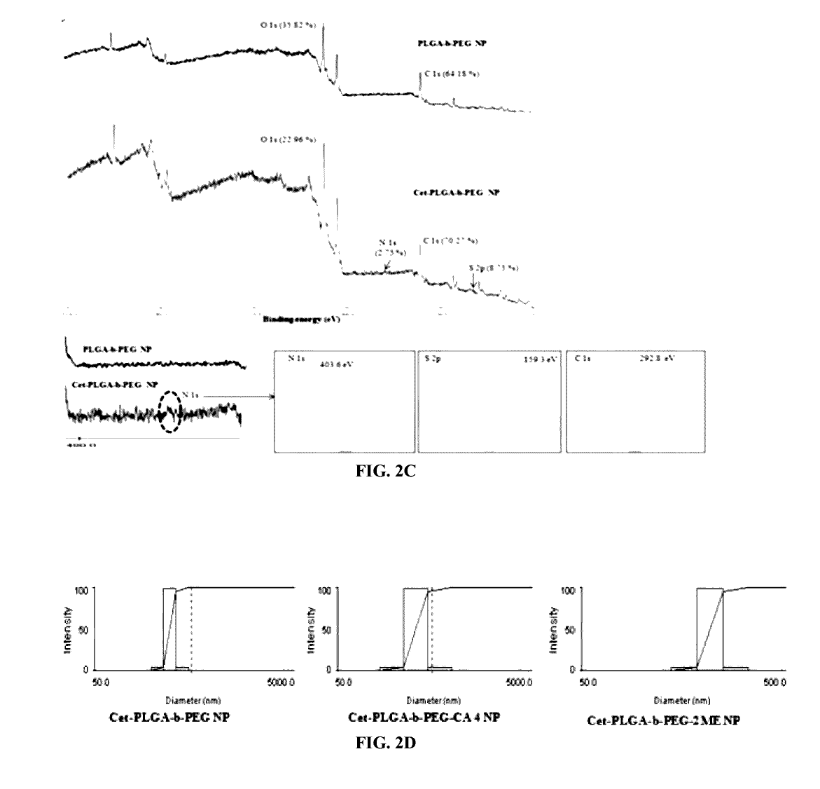

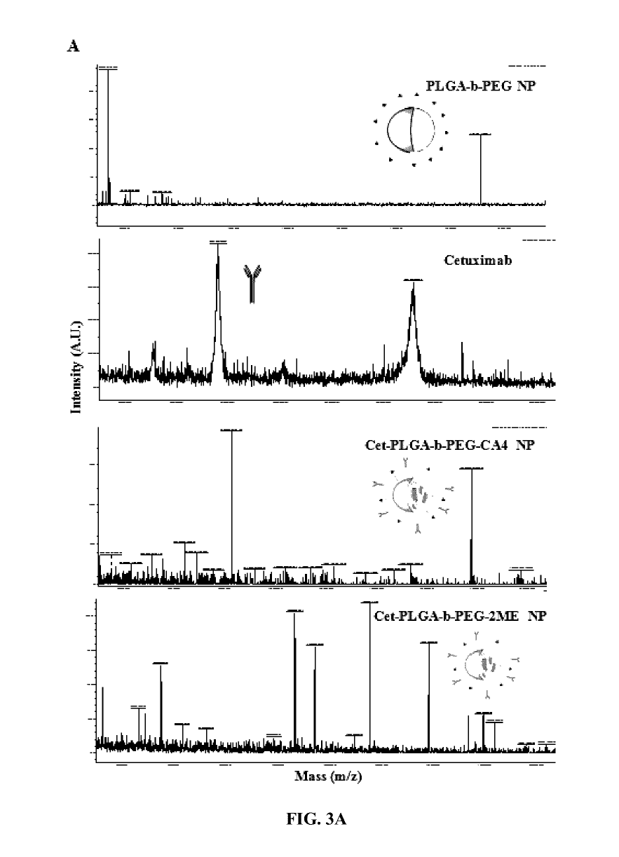

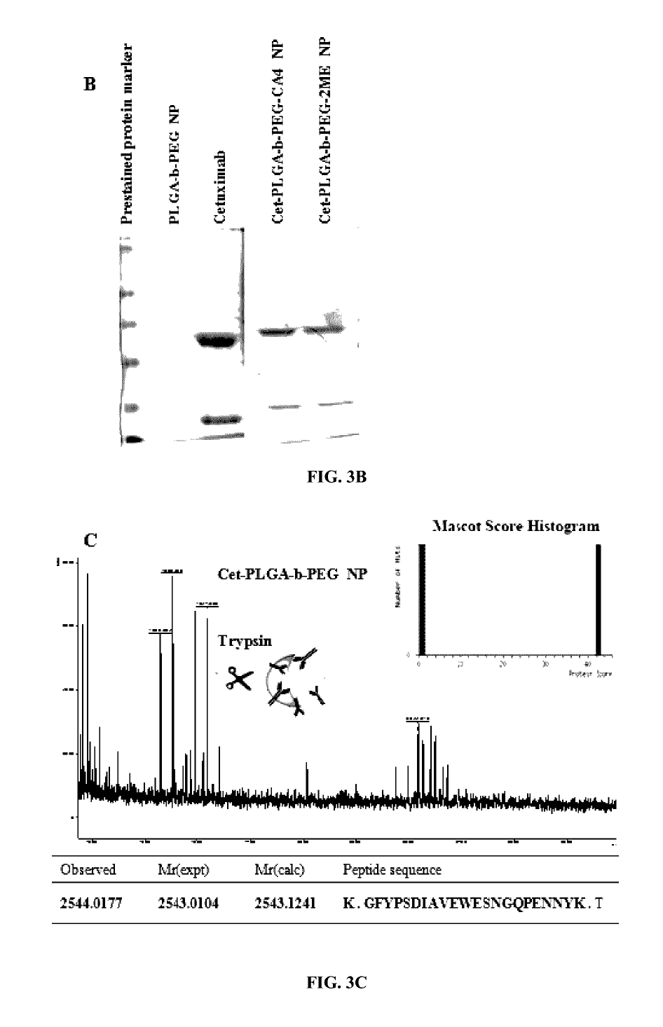

In an embodiment of the present invention, the hydrophilic polymer is covalently conjugated to the hydrophobic polymer core by EDC (1-ethyl-3-(3-dimethylaminopropyl)carbodiimide hydrochloride)/NHS (Sulfo-N-hydroxysuccinimide) coupling and the anti-EGFR targeting moiety is covalently conjugated to the hydrophilic polymer by the same EDC (1-ethyl-3-(3-dimethylaminopropyl)carbodiimide hydrochloride)/NHS (Sulfo-N-hydroxysuccinimide) coupling. The conjugation of the anti-EGFR targeting moiety to the nano-complexes is confirmed by XPS (FIG. 2C), MALDI-TOF MS (FIG. 3A and Table 1) spectra and SDS PAGE (FIG. 3C) analysis.

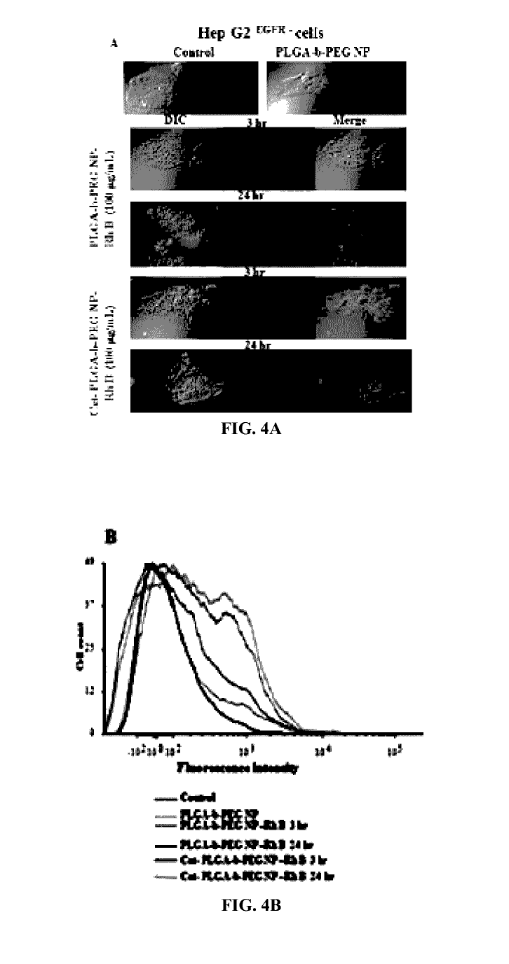

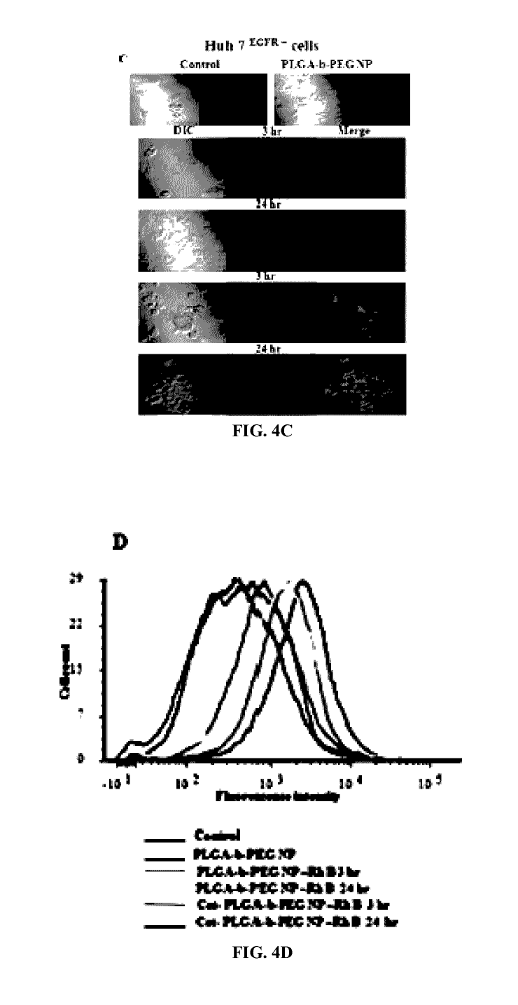

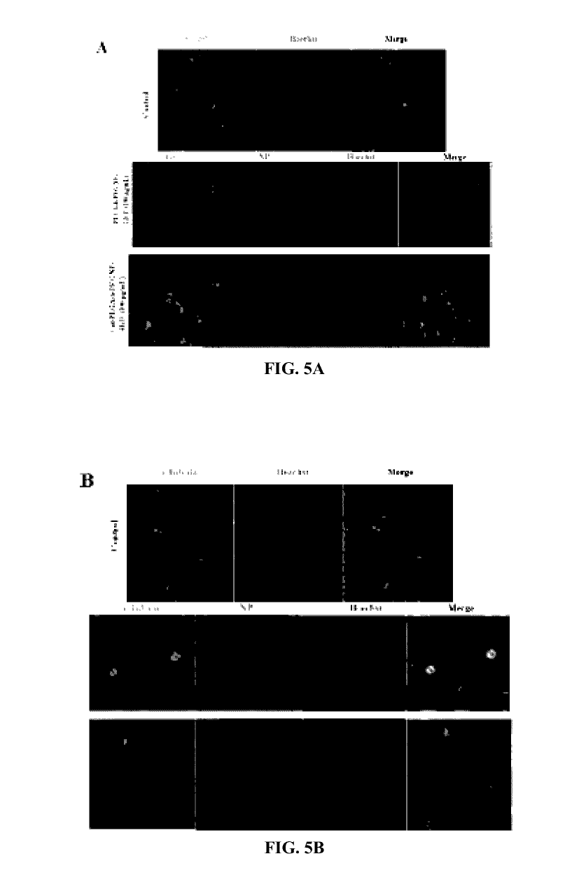

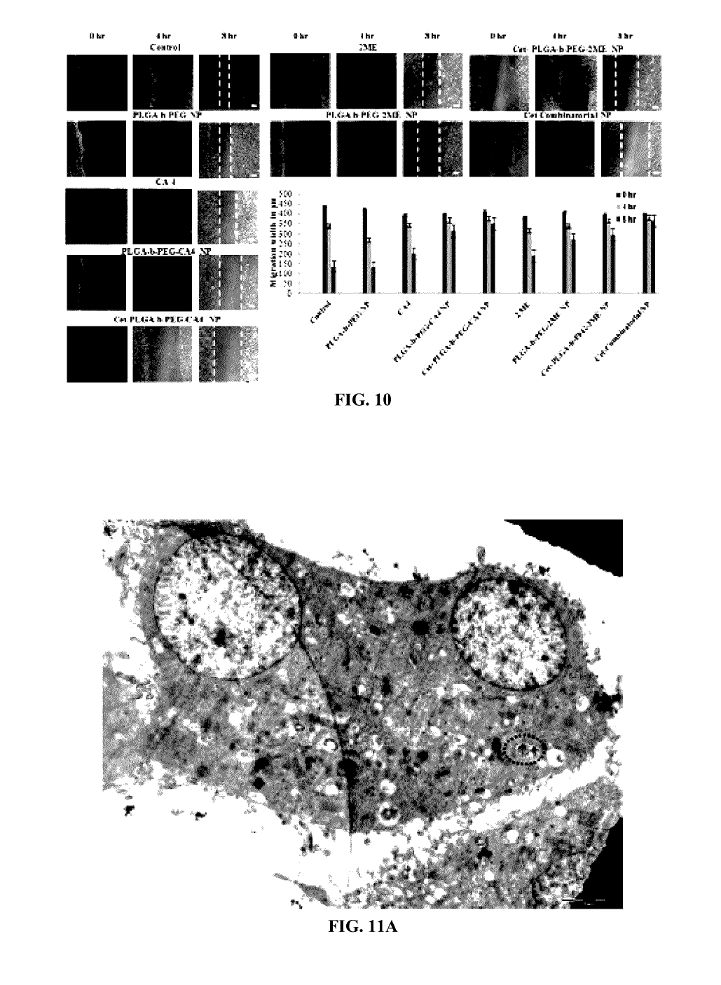

The nano-complexes of the present invention can be used for targeted delivery of drugs in cancers such as but not limited to hepatocellular carcinoma, metastatic colorectal cancer, head and neck cancer. Preferably, the nano-complexes of the present invention act as efficient drug delivery system in EGFR overexpressed cancer cells such as but not limited to human hepatocellular carcinoma (HCC) cells, Huh7 cells, PLC/PRF/5 cells, SK-Hep1 cells, HCCLM3 cells, metastatic hepatocellular carcinoma cell lines, epidermoid carcinoma cell line A431, metastatic colorectal cancer and/or head and neck cancer cell lines. The CLSM, flow cytometry and ultra-structural TEM studies showed the potent internalization of the targeted nano-complexes in Huh7 cells (FIG. 4C, FIG. 4D, FIG. 5B, FIG. 5C and FIG. 11A-FIG. 11C).

Suitable drugs which may be encapsulated or incorporated in the core of the hydrophobic polymer (such as PLGA) of the nano-complex are selected from anti-cancer agents, anti-mitotic agents, anti-angiogenic agents, anti-metastatic agents, anti-proliferative agents or combinations thereof Preferably, the drug may be selected from the group consisting of combretastatin A4 (CA4), 2-methoxyestradiol (2 ME), estramustine, benomyl, vinblastine, colchicine, vincristine, vindesine, vinorelbine, paclitaxel, docetaxel or combinations thereof. The encapsulation efficiency of the drug within the core of the nano-complexes is 30-45% (Table 1).

In an embodiment of the present invention, microtubule destabilizing agents CA4 or 2 ME were encapsulated in PLGA-b-PEG co-polymeric nano-complexes functionalized with chimeric monoclonal antibody cetuximab for targeted delivery to human HCC cells.

The surface morphology of the polymeric nano-complexes of the present invention as analyzed by FEG-TEM and FEG-SEM showed uniform, spherical shaped and smooth surface nano-complexes (FIG. 2B). The FEG-TEM study revealed the architecture of the polymeric nano-complexes as the inner solid core of the hydrophobic polymer (such as PLGA block) encapsulating the drugs. A protective, hydrophilic pegylated outer shell exposed to the aqueous phase forms a corona over the hydrophobic polymer for longer circulation as well as for the conjugation of the anti-EGFR targeting moiety such as Cetuximab.

In an embodiment of the present invention, the nano-complexes of the present invention are nanoparticles.

The average size of the targeted nano-complexes as estimated by the FEG-TEM study is less than 250 nm. The physico-chemical characteristics of the synthesized polymeric nano-complexes are tabulated in Table 1. The polydispersity index of 0.17-0.23 (Table 1) indicated a narrow size distribution of the synthesized nano-complexes, which are well dispersed and non-agglomerated. The targeted nano-complexes have zeta potential value ranging from −10±20 mV.

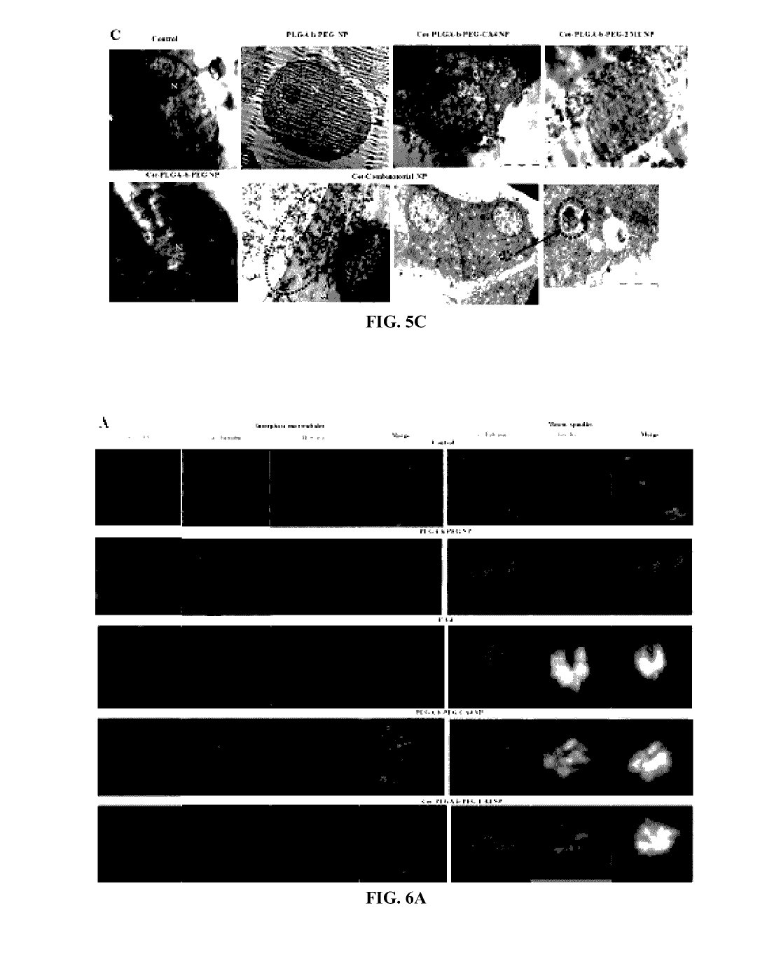

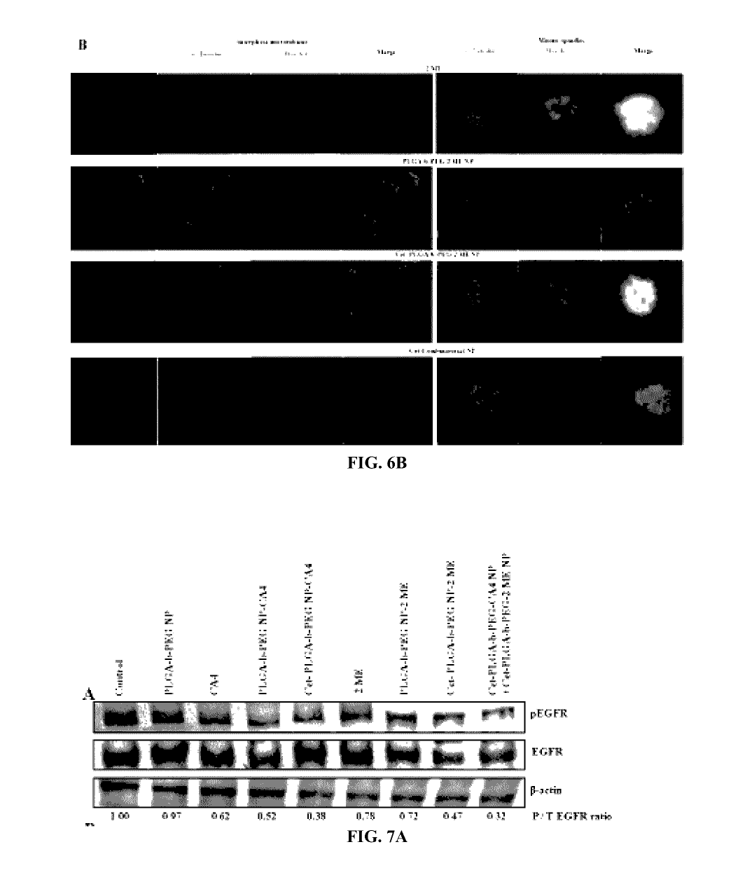

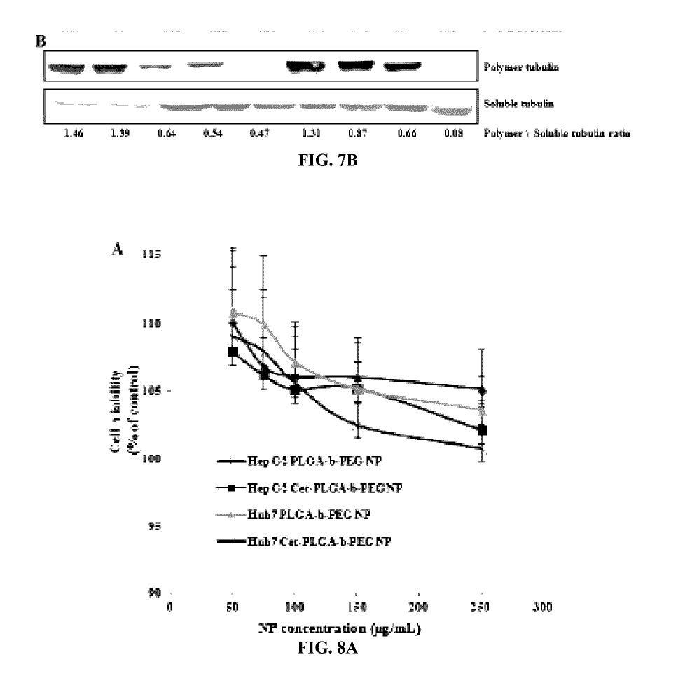

The targeted nano-complexes of the present invention strongly inhibited pEGFR expression and deactivated microtubule polymerization (FIG. 6A, FIG. 6B, FIG. 7A and FIG. 7B). The targeted nano-complexes of the present invention inhibit about 65-85% of EGFR phosphorylation and deactivates about 75-90% of microtubule polymerization.

Immunofluorescence analysis to examine the effect of the synthesized nano-complexes on Histone H3 phosphorylation in cancer cells (FIG. 13) showed that the nano-complex of the present invention strongly inhibits mitotic progression.

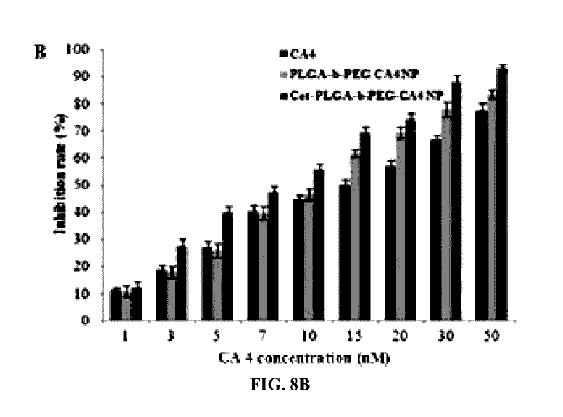

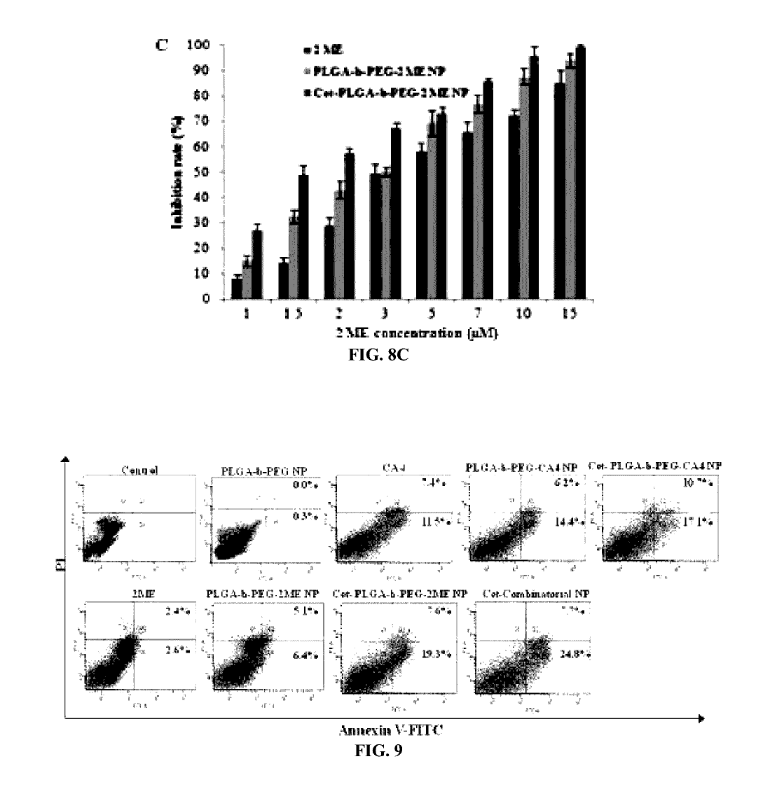

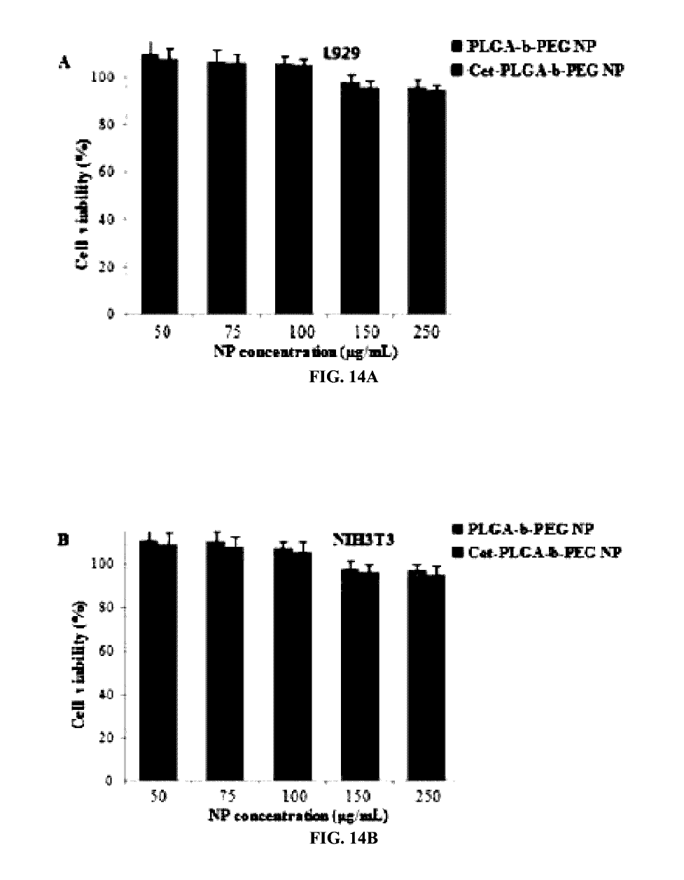

The cytotoxic effect of the synthesized nano-complexes is tested by Sulfo-rhodamine B (SRB assay). The results indicated that the drug encapsulated nano-complexes inhibited Huh7 cell growth in a concentration-dependent manner, thereby leading to enhanced cytotoxicity (Table 2, FIG. 8B and FIG. 8C). The bare nano-complexes were non-cytotoxic to HCC cells at 50, 75, 100, 150 and 250 μg/mL indicating their biocompatibility (FIG. 8A), as well as in normal cells L929 and NIH3T3 (FIG. 14). The IC₅₀ values of the synthesized nano-complexes are represented in Table 2. The IC₅₀ value of the cetuximab targeted PLGA-b-PEG nano-complex encapsulating combretastatin A4 (CA4) drug is 7.9±0.7 nM and the IC₅₀ value of the cetuximab targeted PLEA-b-PEG nano-complex encapsulating 2-methoxyestradiol (2 ME) drug is 1.56±0.15 μM.

The targeted nano-complexes of the present invention also induced apoptosis in cancer cells (FIG. 9) and inhibited cancer cell migration (FIG. 10). The strong anti-migratory effects of the targeted nanocomplexes may prevent the invasion of vascularized cancer cells such as Huh 7 cells.

In an embodiment of the present invention, the targeted nano-complexes of the present invention may also be used for imaging the cells, such as cancer cells. For imaging the cells, the targeted nan-complex of the present invention may optionally comprise a tracking dye such as but not limited to Rhodamine B (Rh B), Fluorescein isothiocyanate (FITC), Tetramethylrhodamine (TRITC), Coumarin 6, Nile red, Rhodamine 123.

In another aspect the present invention also provides a pharmaceutical composition comprising a plurality of the nano-complex as described above and a pharmaceutical acceptable carrier and/or excipients.

The targeted nano-complexes of the present invention may be administered orally, intravenously, intratumorally or subcutaneously.

The present invention also provides a process for the preparation of the afore-mentioned nano-complexes. The process comprises the steps of:

a) conjugating a hydrophobic polymer with a hydrophilic polymer to form a diblock co-polymer;

b) precipitating said diblock co-polymer in ice-cold diethyl ether/methanol solution and drying said diblock co-polymer under vacuum;

c) dissolving said diblock co-polymer and drug in a solvent and emulsifying it in a surfactant solution using an ultrasonic processor to form an oil-in-water emulsion;

d) stirring the resulting emulsion and subjecting it to solvent evaporation using rotary evaporator at reduced pressure to form an aqueous suspension of drug encapsulated nanoparticles;

e) centrifuging said nanoparticles at 20,000×g for 20 minutes at 4° C.;

f) washing said nanoparticles with water and re-suspending it in phosphate buffered saline; and

g) conjugating drug encapsulated nanoparticles with an anti-epidermal growth factor receptor (anti-EGFR) targeting moiety to form the nano-complex.

In an embodiment of the present invention, the hydrophobic polymer is poly (lactic-co-glycolic acid) [PLGA], the hydrophilic polymer is polyethylene glycol (PEG) and the anti-EGFR targeting moiety is cetuximab.

The diblock co-polymer and drug are dissolved in the ratio of 10:1. The solvent which may be used is dimethylformamide (DMF), dichloromethane (DCM), acetone, acetonitrile, ethylacetate, chloroform and the surfactant which may be used is polyvinyl alcohol (PVA) or d-α-tocopheryl polyethylene glycol succinate (TPGS). The conjugation step is performed by EDC/NHS coupling.

The nano-complex obtained at step (g) is further washed and re-suspended in phosphate buffered saline (PBS) and stored at 4° C.

Summing up, the nano-complexes (cetuximab targeted polymeric PLGA-b-PEG nano-complexes) of the present invention strongly accelerated the internalization of the nano-complexes in EGFR overexpressed Huh7 HCC cells, which resulted in significant inhibition of cell proliferation, cell migration and enhanced apoptosis induction in comparison with the non-targeted nano-complexes and free drugs. The targeted nano-complexes of the present invention produced a significant inhibition in the phospho-EGFR expression, enhanced depolymerization of microtubules, increased multinucleation and multipolar spindle formation in cancer cells such as Huh7 cells. In comparison, free CA4 or 2 ME showed less activity at the same concentrations. The combinatorial effects of the targeted nano-complexes, Cet-PLGA-b-PEG-CA4 NP and Cet-PLGA-b-PEG-2 ME NP markedly enhanced the anticancer effects of the antimitotic drugs in Huh7 cells. The cetuximab-functionalized polymeric nano-complexes facilitated the internalization of antimitotic drug CA4 or 2 ME delivery via receptor-mediated endocytosis in EGFR overexpressed HCC cells. Therefore, the nano-complexes of the present invention may be used in designing nanoformulations against clinically relevant EGFR overexpressed HCC therapies.

The present invention will now be more particularly described with reference to the following examples. It is to be understood that these are intended to illustrate the invention and in no manner to limit its scope.

### EXAMPLES

1) Materials:

Combretastastin A4 (CA 4, MW 316.35) and 2-Methoxyestradiol (2 ME, MW 302.41) were purchased from Tocris Bioscience, UK. Poly (D, L-lactide-co-glycolide) (PLGA 50:50, inherent viscosity 0.16-0.24 dl/g, MW 17,000) was provided by Purac Biomaterials, Netherlands. The hetero-functional PEG polymer with a terminal amine and carboxylic acid functional groups (NF₂-PEG₂₀₀₀-COOH) was purchased from Jenkem Technology USA Inc., USA. Polyvinyl alcohol (PVA, MW 1,25,000, viscosity 4%) was obtained from. SD Fine-Chem. Ltd., Mumbai. Cetuximab (Erbitux), a chimeric monoclonal antibody was obtained from Merck Serono, India. 1-ethyl-3-(3-dimethylaminopropyl)carbodiimide hydrochloride (EDC) and N,N-diisopropylethylamine (DIPEA) was obtained from Spectrochem, Mumbai. Dichloromethane (DCM) and Dimethylformamide (DMF) were procured from Merck, Mumbai. Sulfa-N-hydroxysuccinlinide (Sulfo-NHS), Rhodamine B (Rh B), Sulforhodamine B (SRB) reagent and Hoechst 33258 dye were procured from Sigma-Aldrich, USA. Cellulose dialysis membranes (MWCO 12,000) were procured from Hi Media, Mumbai. Annexin V/PI Apoptosis Detection kit was purchased from BD Pharmingen (San Diego, USA). All other reagents were of analytical grade. For cell culture, human HCC Hep G2 cell line with non-EGFR overexpression, normal L929 and NIH3T3 cell lines purchased from National Centre for Cell Science (NCCS, Pune, India), and human HCC Huh7 cell line provided by Indian Institute of Science (IISc, India) with EGFR overexpression were used in the present invention. The Dulbecco's Modified Eagle Medium (DMEM) supplemented with 10% fetal bovine serum (PBS) and 1% penicillin-streptomycin solution obtained from HiMedia, Mumbai was utilized as the cell culture medium. Cells were grown in humidified environment at 37° C. with 5% CO) atmosphere.

2) Synthesis of PLGA-b-PEG Diblock Co-Polymer and Preparation of the PLGA-b-PEG Nano-Complexes:

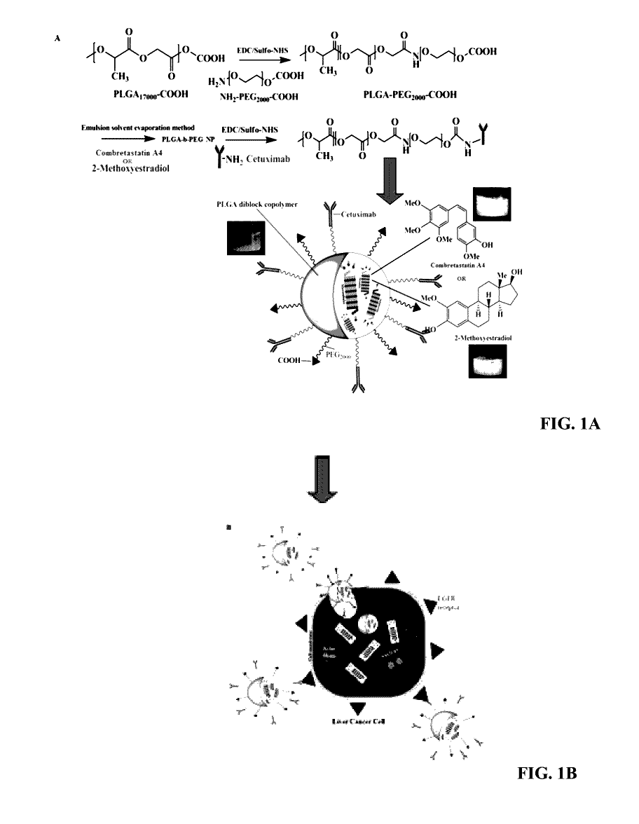

PLGA-b-PEG diblock co-polymer was synthesized by conjugation of PLGA terminated in carboxyl and hydroxyl groups to a heterobifunctional PEG in DCM using NH₂-PEG₂₀₀₀-COOH by carbodiimide/NHS chemistry. PLGA-COOH in DCM was converted to PLGA-NHS by addition of EDC/NHS solution to PLGA-COOH solution with stirring. All the reactions were carried out at room temperature under nitrogen atmosphere. PLGA-NHS was precipitated using ice-cold diethyl ether/methanol as the washing solvent by centrifugation to remove residual EDC/NHS. The washing step was repeated twice. The PLGA-NHS pellet was vaccum dried to remove residual diethyl ether/methanol. PLGA-NHS was then dissolved in DCM followed by addition of NH₂-PEG₂₀₀₀-COOH and N,N-diisopropylethylamine under gentle stirring to form the co-polymer. The resultant co-polymer was purified by precipitating the co-polymer in ice-cold diethyl ether/methanol, removing unreacted PEG through centrifugation (2,700×g for 10 min) three times, and finally dried under vacuum. The percentage yield of the synthesized final product, PLGA-b-PEG diblock co-polymer was calculated using the following formula; % Yield (w/w)=weight of final product/weight of aminated PEG+PLGA-NHS×100%. Nano-complexes were prepared by emulsion-solvent evaporation method (FIG. 1A). Briefly, PLGA-b-PEG diblock co-polymer (10 mg/mL) and antimitotic drugs CA4 or 2 ME (1 mg/mL) were dissolved in DMF and emulsified in 20 ml of 2.5% (w/v) Polyvinyl alcohol aqueous solution using an ultrasonic processor (VCX 130 Watt Vibracell; Sonics) to form an oil-in-water (o/w) emulsion. After the resulting emulsion was stirred, it was subjected to solvent evaporation using rotary evaporator to remove the organic solvent. The aqueous suspension of nanocomplexes was subjected to sterile 0.45 μm syringe filtration to remove the aggregates, and purified by centrifugation at 20,000×g for 20 min at 4° C. by washing with Milli Q water three to four times, which removes all the unentrapped drug/cargo. The resulting purified nano-complexes were resuspended in phosphate buffered saline (PBS, pH 7.4) prior to lyophilization. For cellular uptake studies, Rhodamine B (100 μg/mL) dye loaded nano-complex (PLGA-b-PEG NP-Rh B) was encapsulated as described above.

3) Cetuximab Conjugation of the Activated PLGA-b-PEG Nanocomplexes:

Drug encapsulated nanocomplexes were surface functionalized with cetuximab (Cet), a chimeric monoclonal antibody by EDC-Sulfo-NHS coupling chemistry. Briefly, PLGA-b-PEG nano-complex suspension (10 mg/mL) in PBS was incubated with EDC (400 mM/L) and NHS (100 mM/L) for 20 min with gentle stirring. The NHS-activated nano-complexes were covalently linked to cetuximab (1 mg/mL). The Cet-bound nano-complexes were separated from the unbound Cet and the by-products by centrifugation (35,000×g) for 15 min at 4° C. The centrifugation step was repeated two times after washing with PBS (pH 7.4). The unbound Cet remained in the supernatant. The purified pellet (Cet-nano-complex bioconjugates) was resuspended in PBS (pH 7.4) and stored at 4° C. until further use. Similarly, Cet-PLGA-b-PEG NP-Rh B was formulated for cell uptake studies.

4) Physico-Chemical Characterization of the PLGA-b-PEG Nanocomplexes:

The nuclear magnetic resonance (NMR) spectroscopy was conducted to analyze the presence of PEG binding of the synthesized polymer, NMR spectra of the synthesized PLGA-b-PEG diblock co-polymer were recorded on a Bruker AVANCE-400 NMR spectrometer (Bruker, Switzerland) at 400 MHz using deuterated CDCl₃ as a solvent. The chemical shifts were calibrated using Tetramethylsilane as an internal reference.

The particle size and size distribution of the well-dispersed and nanocomplexes (0.5 mg/mL) diluted in Milli Q water were measured by Dynamic light scattering (DLS, 90Plus, Brookhaven Instruments Corporation, USA) at 25° C., scattering angle of 90° and using a 632 nm red laser. The surface charge of the nanocomplexes was determined by ZetaPlus Zeta-potential analyzer (Brookhaven Instruments Corporation, USA) in Milli Q water at room temperature at pH 7.0. Physical stability of the nanocomplexes when stored at 4° C. as a suspension in PBS (pH 7.4) was examined by evaluating their size distribution and zeta potential over a period of one month. CA4 or 2 ME encapsulation efficiency in the PLGA-b-PEG nano-complexes and Cet-PLGA-b-PEG nano-complexes were measured by UV spectrophotometer (Perkin Elmer, Lambda 25) and calculated using the following formula; Drug encapsulation efficiency (%)=(amount of loaded drug/amount of drug added)×100%.

The size, shape and surface morphology of the nanocomplexes were investigated using the field-emission gun transmission electron microscopy (FEG-TEM, JEOL, JEM-2100 F, Japan) operating at 200 kV. Samples (0.5 mg/mL) were dropped on carbon-coated copper grid, negatively stained with 1% phosphotungstic acid and air dried. For field-emission gun scanning electron microscopy (FEG-SEM, JSM-7600 F, Japan) analysis, the dried nanocomplexes were placed on a stub and coated with gold using a sputter gold coater Auto Fine Coater (JEOL, Tokyo, Japan) prior to SEM application.

The surface chemistry of PLGA-b-PEG NP and Cet-PLGA-b-PEG NP was analyzed using Multilab 2000 X-ray photoelectron spectroscopy (XPS, Thermo VG Scientific) with microfocused monochromatic Al Kα X-ray (1486.6 eV) source. The X-ray angle was 90° C. The pass energy was fixed at 100 eV for the survey scan spectrum covering a binding energy range from 0 to 1200 eV and 25 eV pass energy for the high resolution elemental scan spectrum. The analysis chamber vacuum was <1.5×10⁻⁷ Pa. For peak analysis data, XPS Peak 4.1 software was used.

To assess the formation of nano-complex-bioconjugates viz; intact protein Cet, PLGA-b-PEG NP, Cet-PLGA-b-PEG NP, Cet-PLGA-b-PEG-CA4 NP and Cet-PLGA-b-PEG-2ME NP (50 μg/mL) the mass analysis was performed on a matrix-assisted laser desorption/ionization-time of flight mass spectroscopy (MALDI-TOF MS) AutoFlex Speed Bruker (Bruker Daltonics, Germany) equipped with a smart beam laser (nitrogen laser, 337 nm, 5 ns) as an ion source. The mass spectra were recorded with a 19 kV accelerating voltage, a 100 Hz repetition rate and a linear positive mode with ˜1000 shots. Sinapinic acid (2.5 mg/mL, Bruker, Germany) in 0.1% Trifluoroacetic acid (TEA, Applied Biosystems, Invitrogen, USA) in Water-Acetonitrile (ACN, Sigma-Aldrich, USA) (1:1) was used as a matrix. Internal standard used was Pepmix (Bruker, Germany). Samples and matrix were prepared in 1:1 ratio, 1 μL of the sample mixture were spotted on the stainless-steel standard SCOUT MTP MALDI target plate (Bruker Daltonics, Germany) and air-dried at room temperature for crystallization before loading into the instrument. All the mass spectra were recorded using COMPASS for Flex Series software. In-solution Trypsin (Promega, USA) digestion of Cet-PLGA-b-PEG NP was analyzed by MALDI-TOF MS/MS fingerprinting. Peptide spectra thus obtained were subjected to search the SwissProt database for protein identification using the Mascot server engine online The Cet-PLGA-b-PEG nanocomplexes binding confirmation was also analyzed by gel electrophoresis. After the samples were loaded in SDS-PAGE gel, electrophoresis was carried out in an 80-200V mode for 2 hr. The gel was stained with 0.1% Coomassie Brilliant Blue solution and then destained. A prestained protein marker ladder (4-250 kDa, invitrogen, USA) was also run on the same gel.

5) Cellular Uptake of the PLGA-b-PEC Nanocomplexes and its Intracellular Fate:

For cellular uptake by confocal laser scanning microscopic (CLSM) analysis, Hep G2 and Huh7 cells were seeded on 12 mm glass cover-slips in 24 well plates (Nunc, USA) at 5×10⁴ cells/well density. After reaching 70% confluency, the culture medium was replaced with PLGA-b-PEG NP-Rh B and Cet-PLGA-b-PEG NP-Rh B (100 μg/mL) nanocomplexes for 3 hr and 24 hr of incubation. Images were acquired using CLSM (LSM 510 Meta, Carl Zeiss, Germany) equipped with a 63× oil immersion objective lens and Zeiss LSM Image Browser software. The extents of intracellular uptake of non-targeted vs targeted nanocomplexes by the cells were quantified using flow cytometer BD FACS ARIA special order system (Becton and Dickinson, USA) in terms of median fluorescence intensity and data were analyzed using the FCS Express software.

To study the intracellular distribution, Huh7 cells (5×10⁴ cells/well) were seeded on glass cover-slips in 24 well plates (Nunc, USA) and treated with PLGA-b-PEG NP-Rh B and Cet-PLGA-b-PEG NP-Rh. B (100 μg/mL) for 24 hr and indirect immunofluorescence CLSM analysis was performed.

For intracellular uptake by TEM analysis, Huh7 cells (1×10⁶ cells/mL or more) were incubated in 25 mm tissue culture flasks (Nunc, USA) with 50 μg/mL of Cet-PLGA-b-PEG NP, Cet-PLGA-h-PEG-CA4 NP, Cet-PLGA-b-PEG-2 ME NP and combination of Cet-PLGA-b-PEG-CA4 NP (25 μg/mL)+Cet-PLGA-b-PEG-2 ME NP (25 μg/mL) for 24 hr. Untreated cells served as the control. Briefly, the treated cells were centrifuged, fixed with 2.5% glutaraldehyde, washed with PBS, followed by fixation with 1% osmium tetroxide. Cells were dehydrated in a graded series of alcohol, embedded using DER 332-732 embedding kit (EMS, USA) and polymerized for 48 hr. These resin blocks containing cells were sectioned using an ultramicrotome (Leica Ultracut UCT, Germany) and ultrathin sections (70 nm) were transferred onto 300 mesh Formvar and carbon coated copper grids (EMS, USA). The grids were then processed with uranyl acetate and lead citrate solutions to visualize the cellular ultrastructures. Internalization of the nanocomplexes was visualized under TEM (FEI TECNAI, 12 BioTwin, Netherlands) operating at 80 kV.

To evaluate the effect of nanocomplexes on EGFR and tubulin interactions, Huh7 cells (5×10⁴ cells/well) in 24 well plates were treated with 15 nM of CA4, PLGA-b-PEG-CA4 NP, Cet-PLGA-b-PEG-CA4 NP, 5 μM of 2 ME, PLGA-b-PEG-2 ME NP, Cet-PLGA-b-PEG-2 ME NP and combinatorial Cet-PLEA-b-PEG-CA4 NP+Cet-PLGA-b-PEG-2 ME NP (15 nM+5 μM) for 48 hr. Untreated and bare PLGA-b-PEG NP treated cells served as the negative controls. Cells were then fixed with 3.7% formaldehyde at 37° C. for 30 min and immunostained with primary antibodies anti-EGFR, anti-pEGFR XP rabbit monoclonal antibodies (Cell Signaling Technology Inc., USA), anti-α-Tubulin mouse monoclonal antibody (Signa Aldrich, USA) and anti-pHistone H3 rabbit polyclonal antibody (Ser-10, Santa Cruz Biotechnology, USA) followed by incubation with secondary antibody FITC-conjugated anti-mouse IgG or FITC-conjugated anti-rabbit IgG (Sigma Aldrich, USA) or anti-mouse IgG-Alexa 568 conjugate (Invitrogen, USA). Hoechst 33258 was used for DNA counterstaining and cells were examined under CLSM.

6) Western Blotting:

The EGFR protein expression levels in normal L929, NIH3T3 and HCC Hep G2, Huh7 cell lines were analyzed by Western blotting. For efficacy studies, confluent Huh7 cells in 25 mm tissue culture flasks (Nunc, USA) were incubated with different nanocomplexes for 48 hr. To analyze the level of EGFR phosphorylation, whole cell lysates were prepared by using lysis buffer (20 mM/L Na₂PO₄ (pH 7.4), 150 mM/L NaCl, 1% Triton X-100, 1% aprotinin, 1 mM/L phenylmethylsulfonyl fluoride, 10 g/L leupeptin, 100 mM/L NaF, and 2 mM/L Na3VO4). The cytosolic fraction was collected. In case of microtubule polymerization assay, polymer and soluble tubulin fractions were isolated by incubation with microtubule stabilization buffer (0.1% Triton X-100, 0.1 M MES, pH 6.75, 1 mM MgSO4, 2 mM EGTA, 4 M glycerol) for 10 min at 37° C. The soluble and polymer tubulin fractions were collected. 50 μg of proteins were resolved on SDS-PAGE gel, transferred to nitrocellulose membranes and incubated with primary anti-pEGFR, anti-EGFR XP rabbit monoclonal antibodies (Cell Signaling Technology Inc., USA), anti-α-Tubulin mouse monoclonal antibody (Sigma Aldrich, USA) and β-actin (equal loading control, Cell signaling Technology, Inc, USA), Immunoblots were developed using secondary antibody alkaline phosphatase conjugated-antimouse IgG or -antirabbit IgG (Sigma Aldrich, USA) and the images were processed using the ImageJ software.

7) Cytotoxicity of the PLGA-b-PEG Nanocomplexes:

For cytotoxicity measurements, Hep G2 and Huh7 cells were incubated in 96-well plates (Nunc, USA) at 5×10⁴ cells/well density and then treated with bare PLGA-b-PEG NP, Cet-PLEA-b-PEG NP (50-250 μg/mL), free drug CA4, its nano-complexes (1-50 mM) and free drug 2 ME, its nano-complexes (1-15 μM) for 48 hr. Biocompatibility of the synthesized nanocarriers bare PLGA-b-PEG NP and Cet-PLGA-b-PEG NP (50-250 μg/mL) was also measured in normal L929 and NIH3T3 cells. The standard SRB assay was used to measure the cell viabilities. The absorbance was measured with a 96-well fluorescence plate reader (SPECTRAmax M2, Molecular Devices, USA) equipped with the SOFTmax Pro software (Molecular Devices), at a 550 nun wavelength. IC₅₀ values of the free drugs and all the nanocomplexes were evaluated using OriginPro 8.0 software.

8) Apoptosis Assay:

Annexin V and Propidium iodide (PI) staining detection kit was used to quantify apoptotic cells. Briefly, Huh7 cells (1×10⁵ cells/mL) were seeded in 6-well plates and treated with CA4, PLGA-b-PEG-CA4 NP, Cet-PLGA-b-PEG-CA4 NP, 2 ME, PLGA-b-PEG-2 ME NP and Cet-PLGA-b-PEG-2ME NP at IC₅₀ concentrations for 48 hr. A combination of Cet-PLGA-b-PEG-CA4 NP Cet-PLEA-b-PEG-2ME NP (15 nM+5 μM) was also evaluated. Untreated and bare PLGA-b-PEG NP treated cells served as the negative controls. Cells were washed twice with PBS and resuspended in 400 μL of 1× binding buffer. Then, 5 μL of Annexin V-FITC and PI solution were added to the test samples, incubated for 15 min in dark at room temperature, and analyzed using flow cytometer BD FACS ARIA special order system equipped with the BD FACSDiva. (Becton and Dickinson, USA) software program. The fluorescence of 20,000 cells were gated and counted for each sample.

9) Celt Migration Assay:

Huh7 cells were grown to full confluency in 6 well plates, serum starved (0.2%) for 24 hr and wounds were scratched with a sterile plastic 10 μL pipette tip on the confluent monolayer. Loosened cells were washed with medium, replenished with fresh medium, treated with 15 nM of CA4, PLGA-b-PEG-CA4 NP, Cet-PLGA-b-PEG-CA4 NP, 5 μM of 2 ME, PLGA-b-PEG-2 ME NP, Cet-PLEA-b-PEG-2 ME NP and combinatorial Cet-PLGA-b-PEG-CA4 NP+Cet-PLGA-b-PEG-2 ME NP (15 nM+5 μM), and observed at 0, 4 and 8 hr time intervals under Nikon inverted microscope (ECLIPSE Tiseries, Japan). Images were captured and analyzed using NIS-Elements BR 4.00.00 (Build 764) Imaging software. Untreated cells and bare PLGA-b-PEG NP served as the negative controls. The cell migration rate was expressed as the migration width in μm. Experiments were performed independently in triplicates.

10) Statistical Analysis:

Data were expressed as the mean±SD. Statistical tests were performed with one-way ANOVA using OriginPro 8.0 software. P<0.05 was considered to be statistically significant.

Results

11) Preparation and Physico-Chemical Characterization of the Nanocomplexes:

The conjugation chemistry of PLGA-NH-PEG-COOH co-polymers-antibody via an EDC/NHS reaction and the intracellular trafficking of CA4 or 2 ME encapsulated, and cetuximab surface timctionalized PLGA-h-PEG nanocomplexes into the EGFR overexpressed HCC cells are illustrated in FIG. 1A and FIG. 1B.

The synthesized PLGA-b-PEG &block co-polymer was characterized by ¹H-NMR (CDCl₃, 400 MHz) δ=ppm 5.12-5.29 (m, ((OCH(CH₃)C(O)OCH₂C(O))n-(CH₂CH₂O)m), 4.77-4.88 (m, ((OCH(CH₃)C(O)OCH₂C(O))n-(CH₂CH₂O)m), 3.65 (s, ((OCH(CH₃)C(O)OCH₂C(O)) n-(CH₂CH₂O)m), 1.52-1.64 (d, ((OCH(CH₃)C(O)OCH₂C(O))n-(CH₂CH₂O)m) as the prominent peaks (FIG. 2A), which was in accordance with the previous reports. The yield of the synthesized product, PLGA-b-PEG was found to be 68.2%.

Drug encapsulated nanocomplexes were prepared with an emulsion-solvent evaporation method using 2.5% PVA as the emulsifier and stabilizer (FIG. 1A). The surface morphology of Cet-PLGA-b-PEG NP, Cet-PLGA-b-PEG-CA4 NP and Cet-PLGA-b-PEG-2 ME NP (FIG. 2B) analyzed by FEG-TEM and FEG-SEM showed uniform, spherical shaped and smooth surface nanocomplexes. The architecture of polymeric-coronas are seen in FEG-TEM as the inner hydrophobic solid core of PLGA block encapsulating the hydrophobic microtubule drugs CA4 or 2 ME. A protective, hydrophilic pegylated outer shell exposed to the aqueous phase forming corona for longer circulation as well as for the conjugation of targeting ligands like cetuximab. The average size of the particles was estimated to be 200±20, 196±32 and 245±22 nm for Cet-PLGA-b-PEG NP, Cet-PLGA-b-PEG-CA4 NP and Cet-PLGA-b-PEG-2 ME NP, respectively by FEG-TEM. The sizes of the particles were considered to be suitable for intracellular uptake and in vivo studies. The mean nanoparticle size by FEG-TEM was different than the average hydrodynamic diameter measured by DLS. DLS measurements provides an average hydrodynamic radius of a sphere, which is related to the diffusive particle motion, whereas, the TEM image analysis gives the particles true radius. Using FEG-SEM, the average diameter of the particles were estimated to be 101±16 nm, 112±25 rim and 125±39 nm for Cet-PLGA-b-PEG NP, Cet-PLGA-b-PEG-CA4 NP and Cet-PLGA-b-PEG-2 ME NP, respectively.

The surface analysis of Cet-PLGA-h-PEG NP was performed to confirm the conjugation of cetuximab on the nanocomplex surface by X-ray photoelectron spectroscopy (XPS). The surface composition of the nanocomplexes was evaluated for C1s, O1s, S2p, and N1s signals. As shown in FIG. 2C, no nitrogen element signal was detected in PLGA-b-PEG NP whereas, N 1s (2.75%) was detected in Cet-PLGA-b-PEG NP. Another peculiar feature in Cet-PLGA-b-PEG NP was the presence of S 2p (8.75%) elemental signal and its absence in PLGA-b-PEG NP. This is because cetuximab has a characteristic sulphur element, which confirms its successful conjugation to the nanoparticle surface, as observed in the high resolution elemental scans.

The physico-chemical characteristics and the drug encapsulation efficiency of PLGA-b-PEG-CA4 NP, Cet-PLGA-b-PEG-CA4 NP, PLGA-b-TEG-2 ME NP and Cet-PLGA-b-PEG-2 ME NP are summarized in Table 1 and FIG. 2D. The mean hydrodynamic diameter of the polymeric nanocomplexes in aqueous suspension was estimated to be in the range of 189 to 316 nm by dynamic light scattering and the mean surface charge implicating the nanoparticle stability in dispersion were determined to be in the range of −5.2 to 18.6 mV. The increase in particle diameter and zeta potential of the targeted nanocomplexes in comparison to the non-targeted counterparts could be due to the presence of cetuximab antibody on the surface of the targeted nanopartieles, indicating its successful conjugation. The negative zeta potential was due to the amine terminated PEG chains on particle surface, which may reduce the non-specific interaction between the nanoparticles and cells by the stealth surface effects. The presence of hydrophilic PEG functionalized polymeric nanoparticles allows long-time circulation increasing the half-life, reduces the nanoparticle uptake by non-targeted cells, decreases the non-specific biofouling of nanoparticles and effective against mononuclear-phagocytosis clearance in vivo. The nanoparticle internalization was also evident from the ultrastructural TEM showing potent internalization of Cet-nanocomplexes in Huh7 cells (FIG. 5C). In addition, the nanocomplexes exhibited physical stability over a period of one month on storage in PBS at 4° C. with particle size 200 to 320 nm by DLS and surface charge −7.1 to 20.2 mV showing no significant difference, which may likely to be due to the steric protective effect of PEG on the nanocomplexes surface indicating long-term stability by PEG. The polydispersity index of 0.17-0.23 indicated a narrow size distribution of the synthesized nanocomplexes, which were well dispersed and non-agglomerated. CA4 or 2 ME was encapsulated into PLGA-b-PEG NP and Cet-PLGA-b-PEG NP with an encapsulation efficiency of 30-42% (Table 1).

MALDI-TOF MS fingerprinting was carried out to identify the cetuximab monoclonal antibody adsorbed onto the nanocomplex surface and their respective molecular masses (m/z) are shown in FIG. 3A and Table 1. The molecular mass by MALDI-TOF MS analysis of synthesized PLGA-b-PEG was found to be 19,101.60 m/z. The molecular mass of Cet-PLGA-b-PEG NP was 173595.7 m/z, Cet-PLGA-h-PEG-CA4 NP was 177599.77 m/z and Cet-PLGA-b-PEG-2ME NP was 179644.5 m/z (Table 1). Further, coomassie brilliant blue stained-SDS-PAGE indicated that the conjugation of cetuximab to the nanocomplex surface (FIG. 3B). Protein identification based on the trypsin digested Cet-PLGA-b-PEG NP by MALDI-TOF MS/MS and mascot search revealed the presence of IgG1 peptide with a significant protein score of 42 (FIG. 3C). Thus, the XPS, MALDI-TOF MS spectra and PAGE results were consistent with the confirmation of cetuximab conjugation to the nanocomplexes.

TABLE 1
Physico-chemical parameters of PLGA-b-PEG nanocomplexes

| | | | | | Cet- |
|---|---|---|---|---|---|
| | | Poly- | | Drug | conjugated |
| | Particle | dispersity | Zeta | encapsulation | NP MALDI- |
| | size | index | potential | efficiency | TOF-MS |
| Nanocomplexes | (nm ± SE) | (mean ± SE) | (mV ± SE) | (% ± SE) | (m/z) |
| PLGA-b-PEG NP | 189.0 ± 4.8 | 0.220 ± 0.016 | −9.88 ± 1.50 | — | 19,101.60 |
| PLGA-b-PEG-CA4 NP | 219.4 ± 5.6 | 0.231 ± 0.015 | −5.23 ± 1.08 | 42.12 ± 2.51 | — |
| PLGA-b-PEG-2ME NP | 217.4 ± 7.4 | 0.172 ± 0.027 | 4.66 ± 2.23 | 33.23 ± 3.70 | — |
| Cet-PLGA-b-PEG NP | 291.9 ± 2.3 | 0.213 ± 0.017 | −18.85 ± 4.51 | — | 1,73,595.7 |
| Cet-PLGA-b-PEG-CA4 NP | 316.2 ± 3.1 | 0.207 ± 0.064 | −12.70 ± 0.64 | 40.31 ± 3.40 | 1,77,599.77 |
| Cet-PLGA-b-PEG-2ME NP | 246.9 ± 6.2 | 0.186 ± 0.013 | 18.61 ± 0.72 | 30.40 ± 4.12 | 1,79,644.5 |

Data are the mean of three independent determinations ± SE

12) Uptake Efficiency and Internalization of the Nanocomplexes by HCC Cells:

The uptake of targeted and non-targeted nanocomplexes in EGFR negative Hep G2 and EGFR overexpressed Huh7 cells was examined by CLSM and flow cytometry (FIG. 4). CLSM studies showed the poor uptake capacity of PLGA-b-PEG NP-Rh B and Cet-PLGA-b-PEG NP-Rh B in EGFR negative Hep G2 cells, which exhibited poor red fluorescence intensity in 3 hr and 24 hr (FIG. 4A). As against this, Cet-PLGA-b-PEG NP-Rh B were internalized more efficiently with strong red fluorescence intensity than the non-targeted PLGA-b-PEG NP-Rh B in EGFR overexpressed Huh7 cells (FIG. 4C) in 3 hr and 24 hr. A flow cytometric analysis of Cet-PLGA-b-PEG NP-Rh B also displayed a strong fluorescence signal of 1553±93 and 2378±120, while the non-targeted PLGA-b-PEG NP-Rh B displayed fluorescence signal of 873±35 and 1294±85 in 3 hr and 24 hr, respectively in EGFR overexpressed Huh7 cells (FIG. 4D). The results indicated that the uptake of the targeted nanocomplexes was ˜2.5 folds higher for the targeted nanocomplexes than the non-targeted nanocomplexes (p<0.005). EGFR negative Hep G2 cells showed poor fluorescence signal of 67=12 and 202±22 when treated with PLGA-b-PEG NP-Rh B while Cet-PLGA-b-PEG NP-Rh B showed fluorescence signal of 77±5 and 212±20 in 3 hr and 24 hr, respectively (FIG. 4B). Therefore, Cet-targeted nanocomplexes exhibited ˜15.6 lipid and ˜20.2 fold increased uptake in EGFR overexpressed Huh7 cells in 3 hr and 24 hr, respectively when compared with the EGFR negative Hep G2 cells, indicating its strong uptake efficiency. The flow cytometric results were consistent with the CLSM results indicating an efficient delivery of the PLGA-b-PEG NP-Rh B in the Huh7 cells by cetuximab, which specifically recognized the EGER on the Huh7 cell surface. This indicated an antibody-mediated enhancement in cellular internalization in EGFR overexpressed Huh7 cells. The fluorescence intensity of the untreated control was 53+10 and 393±20, in Hep G2 and Huh7 HCC cells, respectively. The fluorescence intensity was 53±8 and 420±22 for the bare PLGA-b-PEG NP in Hep G2 and Huh7 HCC cells, respectively.

An indirect immunofluorescence analysis by CLSM was conducted to study the intracellular distribution of the nanocomplexes. In Cet-PLGA-b-PEG NP-Rh B treated Huh7 cells, the intense EGFR expression (green) was found to be localized in the plasma membrane followed by an enhancement in the rate of Rh B uptake (red) in the cytoplasm. The findings indicated that cetuximab binds to EGER with high affinity and induced the internalization of EGFR. Some of the nanocomplexes also entered the nucleus at the end of 24 hr in Huh7 cells, which may be due to the capability of cetuximab induced nuclear localization of EGFR. In contrast, non-targeted PLGA-b-PEG NP-Rh B treated Huh7 cells showed poor Rh B uptake efficiency due to the absence of cetuximab antibody (FIG. 5A). The localization of the microtubules (green), the mitotic spindle (green) and the Rh B encapsulated nanocomplexes (red) in the cytoplasm in fixed Huh7 cells are shown in FIG. 5B.

Ultrastructural studies by TEM were conducted to examine the intracellular fate of the nanocomplexes on Huh7 cellular organelles morphology for 24 hr. The control Huh7 cells that were cultured without nanocomplexes showed intact cell membrane and the nucleus (FIG. 5C).

Bare PLGA-b-PEG NP (50 μg/mL) treated cells showed nanoparticles inside the cells with intact cell membrane and nucleus. Bare Cet-PLGA-b-PEG NP (50 μg/mL) were mostly distributed in the cell cytoplasm. Some nanoparticles were adsorbed on the cell membrane. Huh7 cells treated with Cet-PLGA-b-PEG-CA4 NP (50 μg/mL) or Cet-PLGA-b-PEG-2 ME NP (50 μg/mL) depicted disruption of the cell membrane integrity with cetuximab mediated endocytosis enhanced internalization of the nanocomplexes in the cells. Disturbed nuclear envelope with irregularly shaped nucleus, nuclear condensation and fragmentation were the peculiar features. The combinatorial Cet-PLGA-b-PEG-CA4 NP (25 μg/mL)+Cet-PLGA-b-PEG-2 ME NP (25 μg/mL) treated Huh7 cells depicted potent disruption of the cell membrane (oval dotted circle) and have been internalized by the cells (round dotted circle) (FIG. 5C and FIG. 11A-FIG. 11C). The principle mechanism is that a complex of the cetuximab functionalized monoclonal antibody assisted in the binding of a potent microtubule poison to the surface of the liver cancer cells and its subsequent internalization. The efficiency is enhanced by the ligand-mediated transport across the cell membrane. The cytotoxic agent is then released within the tumour cells leading to cell death. Together, the data indicated that cetuximab internalization occurred via the binding to the extracellular domain of the EGFR and that the enhanced cellular targeting uptake efficiency was mediated through the receptor-mediated endocytosis.

13) Effects of Nanocomplexes on the Cellular Dynamics of EGFR in HCC Cells:

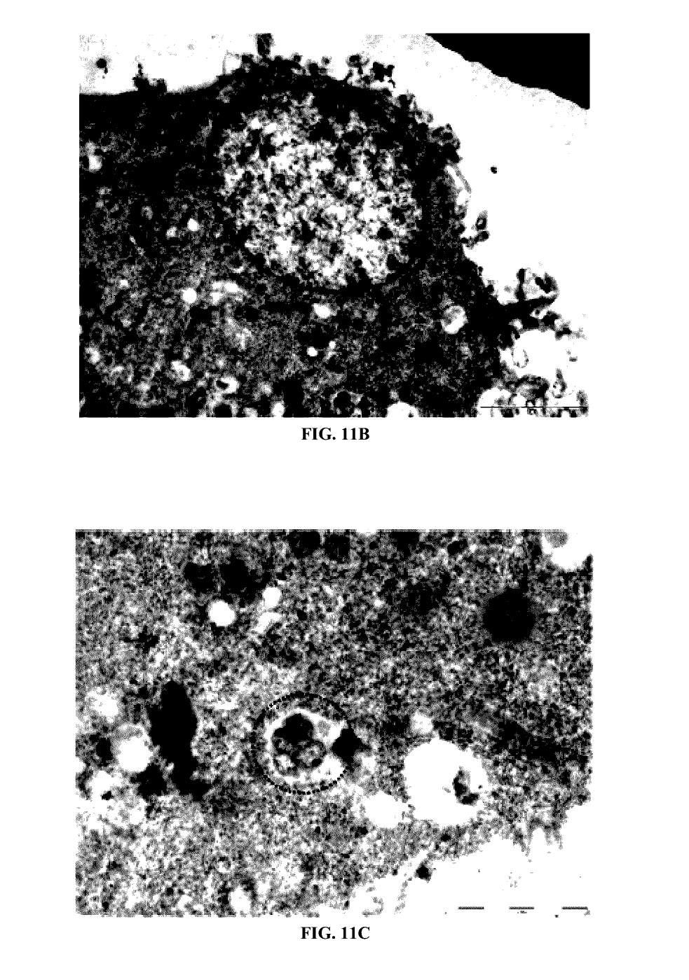

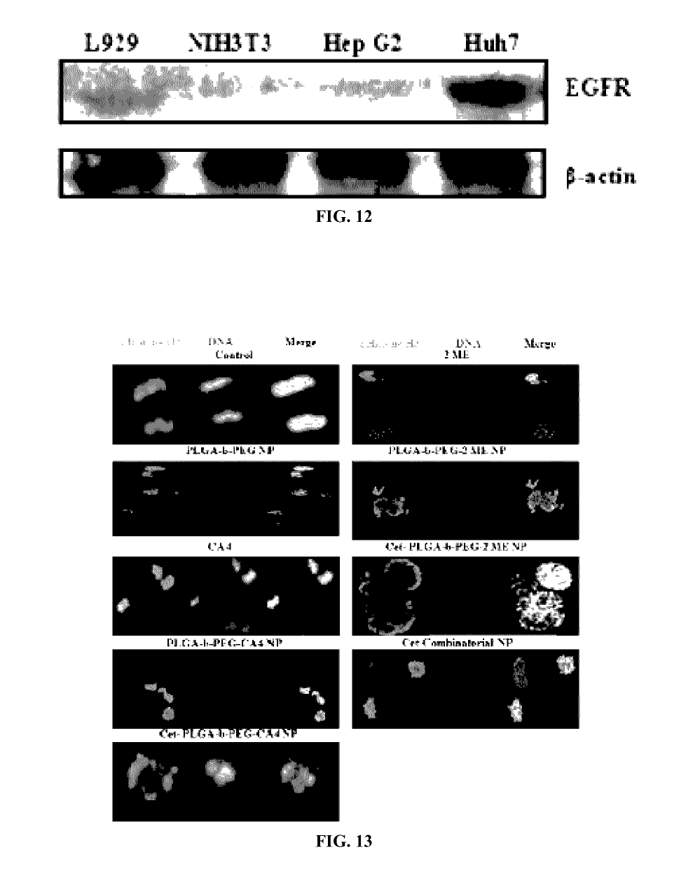

EGFR is found to be overexpressed in human HCC tumours and is associated with an increased metastatic potential and poor prognosis. EGFR-targeting therapeutics holds a promising mode of delivery, internalization and trafficking in cancer cells. The EGFR, protein levels as seen in FIG. 12, by immunoblotting In EGFR overexpressed Huh7 cells showed positive EGFR protein expression in comparison to the EGFR negative Hep G2 cells. Normal L929 and NIH3T3 cells failed to show EGFR protein expression (FIG. 12). The effect of various nanocomplexes on pEGFR expression at indicated concentrations in Huh7 cells after 48 hr were analyzed by CLSM qualitatively. Treatment with 15 nM of CA4, PLGA-b-PEG-CA4 NP or 5 μM of 2 ME, PLGA-b-PEG-2 ME NP moderately affected the expression of pEGFR in Huh7 cells whereas, Cet-PLGA-b-PEG-CA4 NP (15 nM), Cet-PLGA-h-PEG-2 ME NP (5 μM) and combinatorial Cet-PLEA-b-PEG-CA4 NP+Cet-PLEA-h-PEG-2 ME NP (15 nM+5 μM) treatment exhibited strong inhibition in pEGFR expression (FIG. 6A and FIG. 6B). The effects of various nanocomplexes on EGFR phosphorylation levels in Huh7 cells at 48 hr were quantified by immunoblotting. The results showed that Cet-PLGA-h-PEG-CA4 NP and Cet-PLGA-h-PEG-2 ME NP inhibited the activation of EGFR significantly (73% and 65%, respectively) in comparison to the non-targeted nanocomplexes and (61% and 60%, respectively) in comparison to the free drugs as depicted in the phosphorylatedltotal (P/T) EGFR ratio (FIG. 7A). Combinatorial Cet-PLGA-b-PEG-CA4 NP+Cet-PLGA-b-PEG-2 ME NP therapy displayed marked inhibition in pEGFR expression of 84% and 68% in comparison to individual Cet-PLGA-b-PEG-CA4 NP and Cet-PLGA-b-PEG-2 ME NP, respectively. In untreated control cells and bare PLGA-b-PEG NP, the pEGFR expression levels remained nearly unchanged. The western blot results were consistent with the CLSM results which indicated that cetuximab targeted nanocomplexes binds to EGFR with high affinity and induced enhanced EGFR endocytosis internalization leading to EGFR down regulation.

14) Nanocomplexes Perturbed the Microtubule Organization and Stability in HCC Cells:

Microtubules are highly dynamic cytoskeletal polymers and they play a major role in regulating cell division. Dynamic microtubules are involved in spindle formation and chromosomes separation. Mitotic poisons such as CA4 and 2 ME interfere with the mitotic spindle apparatus by perturbing the assembly dynamics of microtubules. These agents are known to perturb the chromosome segregation and to inhibit the cell division at mitosis. Microtubule organization was assessed by qualitative immunofluorescence analysis.

In control untreated Huh? cells and bare PLGA-b-PEG NP treated cells a well-organized microtubule cytoskeleton network showing dense lattice-work, clear normal spindles and chromosomes alignment in a compact manner (FIG. 6A). However, treatment with 15 nM of PLGA-b-PEG-CA4 NP, Cet-PLEA-b-PEG-CA4 NP or 5 μM PLGA-b-PEG-2 ME NP, Cet-PLGA-b-PEG-2 ME NP after 48 hr caused significant depolymerization of microtubules. Many abnormal spindles were found with ball-shaped arrangements of chromosomes and microtubules originating from the single pole extending beyond the chromosomes in these cells. In case of CA4 or 2 ME free drugs in the solution form, though caused microtubule depolymerization but to a lesser extent wherein, some tubulin network was still intact at 15 nM and 5 μM, respectively. The combinatorial Cet-PLGA-b-PEG-CA4 NP+Cet-PLGA-b-PEG-2 ME NP (15 nM+5 μM) therapeutics produced a profound inhibitory effect on microtubules. Multinucleate interphase cells and multipolar abnormal spindles were found to increase significantly (FIG. 6A and FIG. 6B). The mitotic progression was completely arrested. This unusual predominance suggests that the function of mitotic proteins might also be affected. Quantitative immunoblotting analysis too revealed efficient deactivation of microtubule polymerization (i.e. increased soluble tubulin with decreased polymer tubulin expression levels) in Cet-PLGA-b-PEG-CA4 NP (15 nM) and Cet-PLGA-b-PEG-2 NP (5 μM) treated groups (87% and 76%, respectively) in comparison to the non-targeted counterparts and (73% and 50%, respectively) in comparison to the free drugs CA4 or 2 ME at the same concentration at 48 hr (FIG. 7B). This can be further correlated to the qualitative CLSM analysis of interphase microtubule depolymerization (FIG. 6A and FIG. 6B). In Cet-PLGA-b-PEG-CA4 NP+Cet-PLGA-b-PEG-2 ME NP (15 nM+5 μM) combinatorial treatment strongest depolymerization of microtubules was depicted in polymer/soluble tubulin ratio of 0.08. In untreated control cells and bare PLGA-b-PEG NP the tubulin expression levels remained unaffected (FIG. 7B). These dramatic changes may be attributed to the enhanced uptake efficiency of the cetuximab targeted CA4 or/and 2 ME encapsulated PLEA-b-PEG NP for effective delivery into the EGFR overexpressed Huh7 HCC cells and conferred strong lethality. The drugs were released inside the cells from the nanocomplexes, which depolymerized microtubules and induced cell death. The nanocomplexes depolymerized microtubules much more potently than the same concentration of free drugs in the suspension form indicating that the nanocomplex-mediated cetuximab targeted drug delivery strategy has a potential to effectively deliver the microtubule inhibitors.

Phosphorylation of Histone H3 has been known to correlate with mitosis. Therefore, the effect of various nanocomplexes on Histone H3 phosphorylation (Serine 10) in Huh7 cells was examined by immunofluorescence analysis. In untreated control cells, normal mitotic cells with complete localization of pHistone H3 (2+0.2%) in the nucleus and in bare PLGA-b-PEG NP pHistone H3 (2±0.3%) as speckles inside the nucleus was seen. A low level of the expression of pHistone H3 was observed in free drugs CA4 (15 nM, 4±0.3%) or 2 ME (5 μM, 2±0.7%) treated cells implicating less number of antimitotic cells. Increased number of antimitotic cells with aberrant organization of pHistone H3 as flower-like intense chromosome condensations were more prominent in Cet-PLGA-b-PEG-CA4 NP (15 nM, 16±0.5%), Cet-PLGA-b-PEG-2 ME NP (5 μM, 13±0.6%) and combinatorial Cet-PLGA-b-PEG-CA4 NP+Cet-PLGA-b-PEG-2 ME NP (15 nM+5 μM, 21±0.3%) treatment groups after 48 hr (FIG. 13). A significant proportion of cells were specifically arrested in mitosis and failed to progress through the cell cycle. PLGA-b-PEG-CA4 NP (15 nM, 7±0.6%) and PLGA-b-PEG-2 ME NP (5 μM, 4±0.8%) treated cells too depicted condensed chromosomes. The results indicated increased number of pHistone H3 positive Huh7 cells elicited by the targeted delivery of nanocomplexes in comparison to the non-targeted counterparts, implicated its ability to inhibit the mitotic progression effectively.

15) Cytotoxic Effects of the Nanocomplexes:

The sensitivity of Hep G2 and Huh7 HCC cells to CA4 and its nanocomplexes (1-50 nM) as well as 2 ME and its nanocomplexes (1-15 μM) treatments were tested by Sulforbodamine B (SRB) assay after 48 hr incubation. These formulations inhibited Huh7 cell growth in a concentration-dependent manner. The bare PLGA-b-PEG NP and Cet-PLGA-b-PEG NP were non-cytotoxic to HCC cells at 50, 75, 100, 150 and 250 μg/mL indicating their biocompatibility (FIG. 8A), as well as in normal cells L929 and NIH3T3 as expected (FIG. 14). Cetuximab by itself is unable to induce cell growth suppression in vitro as they were not cytotoxic but, require an antibody-dependent cellular cytotoxicity (ADCC, immune response). This finding is consistent with the non-cytotoxic nature of Cet-PLGA-b-PEG NP. The IC₅₀ values are represented in Table 2. In Huh7 cells, IC₅₀ of PLGA-b-PEG-CA4 NP was significantly different (p<0.05) in comparison to CA4. No significant difference in the IC₅₀ between 2 ME and PLGA-h-PEG-2 ME NP was observed, Strong anti-proliferative effects (p<0.005) were exerted by Cet-PLGA-b-PEG-CA4 NP (IC₅₀=7.9 nM) or Cet-PLGA-b-PEG-2 ME NP (IC₅₀=1.56 μM) in comparison to the free drugs CA4 (IC₅₀=14.9 nM) or 2 ME (IC₅₀=3.5 μM) as well as the non-targeted nanocomplexes PLGA-b-PEG-CA4 NP (IC₅₀=11.1 nM) and PLGA-b-PEG-2 ME NP (IC₅₀=2.88 μM) depicting the efficiency of targeting moiety, cetuximab functionalization in enhanced uptake by Huh7 cells leading to enhanced cytotoxicity (Table 2, FIG. 8B and FIG. 8C). In case of Hep G2 cells, CA4 and PL:GA-b-PEG-CA4 NP showed cytotoxic activity at IC₅₀>5 μM higher concentration. Cet-PLGA-b-PEG-CA4 NP failed to show any significant growth inhibition with similar growth inhibitory rate as its non-targeted PLGA-b-PEG-CA4 NP counterpart. This may be due to the negative EGFR expression in Hep G2 cells as evident from the CLSM and flow cytometric cell uptake analysis (FIG. 4A and FIG. 4B). Similar results were observed in case of 2 ME (IC₅₀ 5 μM) and PLGA-b-PEG-2 ME NP (IC₅₀ 10 μM) exhibiting cytotoxicity at a higher concentration. Cet-PLGA-b-PEG-2 ME NP failed to exhibit any growth inhibitory difference in comparison to its non-targeted counterpart concluding that the treatments were insensitive to Rep G2 cells. This study illustrated that the delivery of CA4 or 2 ME via cetuximab targeted nanocomplexes is beneficial in enhancing the cytotoxic efficiency against EGFR overexpressing Huh7 HCC cancer cells. The results indicated that cetuximab nanocomplexes exerted EGFR-targeting specificity in comparison to the EGFR negative Rep G2 cells. The cytotoxicity efficacy study is coherent with the cellular uptake study between the two cell lines.

TABLE 2
IC₅₀ of various
PLGA-b-PEG nanocomplexes
after 48 hr incubation Huh7 cells

| Nanocomplexes | IC₅₀ |
|---|---|
| CA4 (nM) | 14.9 ± 1.6 |
| 2 ME (μM) | 3.5 ± 0.5 |
| PLGA-b-PEG NP | — |
| PLGA-b-PEG-CA4 NP (nM) | 11.1 ± 0.6* |
| PLGA-b-PEG-2 ME NP (μM) | 2.88 ± 0.3 |
| Cet-PLGA-b-PEG NP | — |
| Cet-PLGA-b-PEG-CA4 | 7.9 ± 0.7** |
| NP (nM) | |
| Cet-PLGA-b-PEG-2 ME | 16 ± 1.5 |
| NP (μM) | |

Data are the mean ± SD of three independent experiments; *P < 0.05 significantly different from CA4, **P < 0.005 significantly different from PLGA-b-PEG-CA4 NP, CA4, PLGA-b-PEG-2 ME NP and 2 ME groups

16) Nanocomplexes Induced Apoptosis in Huh7 Cells:

Dysregulation of the apoptosis machinery impinges the normal functioning of cells and promotes tumorigenesis. Hence, the induction of apoptotic cell death is a crucial strategy for cancer chemotherapy. The effects of the nanocomplexes on apoptosis were evaluated in Huh7 cells for 48 hr using Annexin V/PI assay at IC₅₀ concentrations by flow cytometry. Treatment of cells with the various nanocomplexes induced apoptosis more potently as compared to that of free drugs (FIG. 9). However, the Cet-PLGA-b-PEG-CA4 NP+Cet-PLGA-b-PEG-2 ME NP (15 nM+5 μM) combinatorial therapy showed a marked apoptotic effect. The order of induction of apoptosis was Cet-combinatorial NP (24.8%)>Cet-PLGA-b-PEG-CA4 NP (17.1%)>PLGA-b-PEG-CA4 NP (14.4%)>CA4 (11.5%) or Cet-PLGA-b-PEG-2 ME NP (19.3%)>PLGA-b-PEG-2 ME NP (6.4%)>2 ME (2.6%). Furthermore, the efficient uptake of Cet-PLGA-h-PEG-CA4 NP or Cet-PLGA-b-PEG-2 ME NP as well as combinatorial therapy in the Huh7 cells significantly enhanced the cell death due to the accelerated internalization process by cetuximab.

17) Cettiximab Functionalized Nanocomplexes Potently Blocked Huh7 Cell Migration:

HCC are highly vascularized tumours. Cell migration is a critical factor during the new blood vessel formation. Tumour cells show increased metastasis and invasive properties with increased cell migration. The effects of different nanocomplexes on wound closure by scratch assay were evaluated. The nanocomplexes demonstrated differential rates of Huh7 cell migration at 0 hr, 4 hr and 8 hr (FIG. 10). Wound closure was observed in the control and PLGA-b-PEG NP groups after 8 hr of incubation. A significant inhibition in cell migration rate was observed on treatment of 15 nM Cet-PLGA-b-PEG-CA4 NP (3.8 fold, p<0.01) vs CA4, Cet-PLGA-b-PEG-CA4 NP vs PLGA-b-PEG-CA4 NP (1.2 fold, p<0.05) and PLGA-b-PEG-CA4 NP (3.2 fold, p<0.05) vs CA4 treatment groups at 8 hr. Similarly, a significant inhibition in cell migration rate was observed on treatment of 5 μM PLGA-b-PEG-2 ME NP (2.3 fold, p<0.008) vs 2 ME, Cet-PLGA-b-PEG-2 ME NP (2.9 fold, p<0.005) vs 2 ME and (1.2 fold, p<0.08) vs PLGA-b-PEG-2 ME NP groups at 8 hr. The combinatorial Cet-PLGA-b-PEG-CA4 NP+Cet-PLGA-b-PEG-2 ME NP (15 nM+5 μM) treatment showed strongest anti-migratory effect (p<0.0001) in Huh7 cells wherein a larger wound area remained uncovered in comparison to the free drugs CA 4 (3.9 fold) or 2 ME (4.3 fold) treatment at 8 hr. Also, the combinatorial treatment showed potent inhibition in cell migratory effects (p<0.01) in comparison to individual. Cet-PLGA-b-PEG-CA4 NP (1.1 fold) and Cet-PLGA-b-PEG-2 ME NP (1.5 fold) at 8 hr. The results indicated that the cetuximab-functionalized blocked the Huh7 cell migration more efficiently than CA 4 or 2 ME alone. The strong anti-migratory effects of the cetuximab targeted nanocomplexes may prevent the invasion of vascularized Huh7 cells, a crucial factor for antiangiogenesis.

1) A nano-complex comprising:
a hydrophobic polymer core encapsulating at least one drug inside the core;
a hydrophilic polymer conjugated to said core; and
an anti-epidermal growth factor receptor (anti-EGFR) targeting moiety conjugated to the hydrophilic polymer.

2) The nano-complex as claimed in claim 1, wherein the hydrophobic polymer is poly (lactic-co-glycolic acid) [PLGA], 50/50 PLGA, 65/35 PLGA, 75/25 PLGA, 85/15 PLGA or its derivatives.

3) The nano-complex as claimed in claim 1, wherein the hydrophilic polymer is polyethylene glycol (PEG).

4) The nano-complex as claimed in claim 1, wherein the anti-EGFR targeting moiety is an anti-EGFR antibody, an anti-EGFR antibody-like molecule, an anti-EGFR Fe portion, an anti-EGFR Fab, an anti-EGFR Fab₂, an anti-EGFR ScFv, an anti-EGFR single domain antibody or an anti-EGFR nano-body or an anti-EGFR ligand antibody or an anti-EGFR ligand antibody fragment or its variants or fragments thereof.

5) The nano-complex as claimed in claim 4, wherein the anti-EGFR targeting moiety is cetuximab, pinitumumab, zalutumumab, niniotuzumab, matuzumab or its variants and/or its fragments.)

6) The nano-complex as claimed in claim 1, wherein the drug is an anti-cancer agent, an anti-mitotic agent, an anti-angiogenic agent, an anti-metastatic agent, an anti-proliferative agent or combinations thereof.

7) The nano-complex as claimed in claim 1, wherein the drug is combretastatin A4 (CA4), 2-methoxyestradiol (2 ME), estramustine, benomyl, vinblastine, colchicine, vincristine, vindesine, vinorelbine, paclitaxel, docetaxel or combinations thereof.

8) The nano-complex as claimed in claim 1, wherein the hydrophilic polymer is covalently conjugated to the hydrophobic polymer core by EDC (1-ethyl-3-(3-dimethylaminopropyl)carbodiimide hydrochloride)/NHS (Sulfo-N-hydroxysuccinimide) coupling.

9) The nano-complex as claimed in claim 1, wherein the anti-EGFR targeting moiety is covalently conjugated to the hydrophilic polymer by EDC (1-ethyl-3-(3-dimethylaminopropyl)carbodiimide hydrochloride)/NHS (Sulfo-N-hydroxysuccinimide) coupling.

10) The nano-complex as claimed in claim 1, wherein the hydrophilic polymer forms a corona on the hydrophobic polymer core.

11) The nano-complex as claimed in claim 1, wherein the average size of the nano-complex is less than 250 nm.

12) The nano-complex as claimed in claim 1, wherein said nano-complex has a zeta potential value ranging from −10±20 mV.

13) The nano-complex as claimed in claim 1, wherein said nano-complex deactivates about 75-90% of microtubule polymerization.

14) The nano-complex as claimed in claim 1, wherein said nano-complex inhibits about 65-85% of EGFR. phosphorylation.

15) The nano-complex as claimed in claim 1, wherein the IC₅₀ value of cetuximab targeted PLGA-b-PEG nano-complex encapsulating combretastatin A4 (CA4) drug is about 7.9±0.7 nM.

16) The nano-complex as claimed in claim 1, wherein the IC₅₀ value of cetuximab targeted PLGA-b-PEG nano-complex encapsulating 2-methoxyestradiol (2 ME) drug is about 1.56±0.15 μM.

17) The nano-complex as claimed in claim 1, wherein the nano-complex optionally comprises a tracking dye; wherein the tracking dye is selected from the group consisting of Rhodamine B (Rh B), Fluorescein isothiocyanate (FITC), Tetramethylrhodamine (Tanc), Coumarin 6, Nile red, Rhodamine 123.

18) A pharmaceutical composition comprising a plurality of nano-complex as claimed in claim 1 and a pharmaceutical acceptable carrier and/or excipients.

19) A process for the preparation of nano-complex comprising the steps of:
a) conjugating a hydrophobic polymer with a hydrophilic polymer to form a diblock co-polymer;
h) precipitating said diblock co-polymer in ice-cold diethyl ether/methanol solution and drying said diblock co-polymer under vacuum;
c) dissolving said diblock co-polymer and drug in a solvent and emulsifying it in a surfactant solution using an ultrasonic processor to form an oil-in-water emulsion;
d) stirring the resulting emulsion and subjecting it to solvent evaporation using rotary evaporator to form an aqueous suspension of drug encapsulated nanoparticles;
e) centrifuging said nanoparticles at 20,000×g for 20 minutes at 4° C.;
f) washing said nanoparticles with water and re-suspending it in phosphate buffered saline; and
g) conjugating drug encapsulated nanoparticles with an anti-epidermal growth factor receptor (anti-EGFR) targeting moiety to form the nano-complex.

20) The process as claimed in claim 19, wherein said diblock co-polymer and drug are dissolved in the ratio of 10:1.

21) The process as claimed in claim 19, wherein the hydrophobic polymer is poly (lactic-co-glycolic acid) [PLGA], the hydrophilic polymer is polyethylene glycol (PEG) and the anti-EGFR targeting moiety is cetuximab.

22) The process as claimed in claim 19, wherein the solvent is dimethylforamide (DMF), dichloromethane (DCM), acetone, acetonitrile, ethylacetate, chloroform and the surfactant is polyvinyl alcohol (PVA) or d-α-tocopheryl polyethylene glycol succinate (TPGS).

23) The process as claimed in claim 19, wherein step of conjugation is performed by EDC/NHS coupling.

24) The process a claimed in claim 18, wherein the nano-complex obtained at step (g) is further washed and re-suspended in phosphate buffered saline (PBS) and stored at 4° C.

25) A method of treating cancer or inhibition of cell proliferation of cancer comprising administering to the subject an effective amount of nano-complex as claimed in claim 1.

26) A method of inhibiting the expression of EGFR phosphorylation and microtubule polymerization in cancer cells comprising administering to the subject an effective amount of nano-complex as claimed in claim 1.
